(19) United States
(12) Patent Application Publication
(10) Pub. No.: US 2019/0370580 A1
(43) Pub. Date: Dec. 5, 2019

(54) DRIVER MONITORING APPARATUS, DRIVER MONITORING METHOD, LEARNING APPARATUS, AND LEARNING METHOD

(71) Applicant: OMRON Corporation, Kyoto-shi, KYOTO (JP)

(72) Inventors: Hatsumi AOI, Kyotanabe-shi (JP); Koichi KINOSHITA, Kyoto-shi (JP); Tomoyoshi AIZAWA, Kyoto-shi (JP); Tadashi HYUGA, Hirakata-shi (JP); Tomohiro YABUUCHI, Kizugawa-shi (JP); Mei UETANI, Uji-shi (JP)

(73) Assignee: OMRON Corporation, Kyoto-shi, KYOTO (JP)

(21) Appl. No.: 16/484,480

(22) PCT Filed: May 26, 2017
(86) PCT No.: PCT/JP2017/019719

§ 371 Date: Aug. 8, 2019

(30) Foreign Application Priority Data
Mar. 14, 2017 (JP) 2017-049250

(51) Int. Cl. G06K 9/00; G06K 9/62; G08G 1/0962; G06N 3/08; A61B 5/18; A61B 5/16; A61B 5/00; B60W 40/08

(52) U.S. Cl. CPC G06K 9/00845; G06K 9/00315; G06K 9/6256; G08G 1/09626; G06N 3/08; B60W 2420/42; A61B 5/165; A61B 5/7267; A61B 5/746; A61B 5/163; B60W 40/08; A61B 5/18

(57) ABSTRACT

A driver monitoring apparatus according to an aspect of the present invention includes: an image obtaining unit that obtains a captured image from an imaging apparatus arranged so as to capture an image of a driver seated in a driver seat of a vehicle; an observation information obtaining unit that obtains observation information regarding the driver, the observation information including facial behavior information regarding behavior of a face of the driver; and a driver state estimating unit that inputs the captured image and the observation information to a trained learner that has been trained to estimate a degree of concentration of the driver on driving, and obtains, from the learner, driving concentration information regarding the degree of concentration of the driver on driving.

### TECHNICAL FIELD

The present invention relates to a driver monitoring apparatus, a driver monitoring method, a learning apparatus, and a learning method.

### BACKGROUND ART

Techniques have been developed recently for monitoring the state of a driver to prevent automobile traffic accidents caused by falling asleep, sudden changes in physical condition, and the like. There has also been an acceleration in trends toward automatic driving technology in automobiles. In automatic driving, steering of the automobile is controlled by a system, but given that situations may arise in which the driver needs to take control of driving from the system, it is necessary during automatic driving to monitor whether or not the driver is able to perform driving operations. The need to monitor the driver state during automatic driving has also been confirmed at the intergovernmental meeting (WP29) of the United Nations Economic Commission for Europe (UN-ECE). In view of this as well, development is underway for technology for monitoring the driver state.

Examples of technology for estimating the driver state include a method proposed in Patent Literature 1 for detecting the real degree of concentration of a driver based on eyelid movement, gaze direction changes, or small variations in the steering wheel angle. With the method in Patent Literature 1, the detected real degree of concentration is compared with a required degree of concentration that is calculated based on vehicle surrounding environment information to determine whether the real degree of concentration is sufficient in comparison with the required degree of concentration. If the real degree of concentration is insufficient in comparison with the required degree of concentration, the traveling speed in automatic driving is lowered. The method described in Patent Literature 1 thus improves safety during cruise control.

As another example, Patent Literature 2 proposes a method for determining driver drowsiness based on mouth opening behavior and the state of muscles around the mouth. With the method in Patent Literature 2, if the driver has not opened their mouth, the level of driver drowsiness is determined based on the number of muscles that are in a relaxed state. In other words, according to the method in Patent Literature 2, the level of driver drowsiness is determined based on a phenomenon that occurs unconsciously due to drowsiness, thus making it possible to raise detection accuracy when detecting that the driver is drowsy.

As another example, Patent Literature 3 proposes a method for determining driver drowsiness based on whether or not the face orientation angle of the driver has changed after eyelid movement. The method in Patent Literature 3 reduces the possibility of erroneously detecting a downward gaze as a high drowsiness state, thus raising the accuracy of drowsiness detection.

As another example, Patent Literature 4 proposes a method for determining the degree of drowsiness and the degree of inattention of a driver by comparing the face image on the driver's license with a captured image of the driver. According to the method in Patent Literature 4, the face image on the license is used as a front image of the driver in an awake state, and feature quantities are compared between the face image and the captured image in order to determine the degree of drowsiness and the degree of inattention of the driver.

As another example, Patent Literature 5 proposes a method for determining the degree of concentration of a driver based on the gaze direction of the driver. Specifically, according to the method in Patent Literature 5, the gaze direction of the driver is detected, and the retention time of the detected gaze in a gaze area is measured. If the retention time exceeds a threshold, it is determined that driver has a reduced degree of concentration. According to the method in Patent Literature 5, the degree of concentration of the driver can be determined based on changes in a small number of gaze-related pixel values. Thus, the degree of concentration of the driver can be determined with a small amount of calculation.

### CITATION LIST

### Patent Literature

Patent Literature 1: JP 2008-213823A

Patent Literature 2: JP 2010-122897A

Patent Literature 3: JP 2011-048531A

Patent Literature 4: JP 2012-084068A

Patent Literature 5: JP 2014-191474A

### SUMMARY OF INVENTION

### Technical Problem

The inventors of the present invention found problems such as the following in the above-described conventional methods for monitoring the driver state. In the conventional methods, the driver state is estimated by focusing on changes in only certain portions of the driver's face, such as changes in face orientation, eye opening/closing, and changes in gaze direction. There are actions that are necessary for driving such as turning one's head to check the surroundings during right/left turning, looking backward for visual confirmation, and changing one's gaze direction in order to check mirrors, meters, and the display of a vehicle-mounted device, and such behaviors can possibly be mistaken for inattentive behavior or a reduced concentration state. Also, in the case of reduced attention states such as drinking or smoking while looking forward, or talking on a mobile phone while looking forward, there is a possibility that such states will be mistaken for normal states. In this way, given that conventional methods only use information that indicates changes in portions of the face, the inventors of the present invention found that there is a problem that such methods cannot accurately estimate the degree to which a driver is concentrating on driving with consideration given to various states that the driver can possibly be in.

One aspect of the present invention was achieved in light of the foregoing circumstances, and an object thereof is to provide technology for making it possible to estimate the degree concentration of a driver on driving with consideration given to various states that the driver can possibly be in.

### Solution to Problem

The following describes configurations of the present invention for solving the problems described above.

A driver monitoring apparatus according to one aspect of the present invention includes: an image obtaining unit configured to obtain a captured image from an imaging apparatus arranged so as to capture an image of a driver seated in a driver seat of a vehicle; an observation information obtaining unit configured to obtain observation information regarding the driver, the observation information including facial behavior information regarding behavior of a face of the driver; and a driver state estimating unit configured to input the captured image and the observation information to a trained learner that has been trained to estimate a degree of concentration of the driver on driving, and configured to obtain, from the learner, driving concentration information regarding the degree of concentration of the driver on driving.

According to this configuration, the state of the driver is estimated with use of the trained learner that has been trained to estimate the degree of concentration of the driver on driving. The input received by the learner includes the observation information, which is obtained by observing the driver and includes facial behavior information regarding behavior of the driver's face, as well as the captured image, which is obtained from the imaging apparatus arranged so as to capture images of the driver seated in the driver seat of the vehicle. For this reason, the state of the driver's body can be analyzed based on not only the behavior of the driver's face, but also based on the captured image. Therefore, according to this configuration, the degree of concentration of the driver on driving can be estimated with consideration given to various states that the driver can possibly be in. Note that the observation information may include not only the facial behavior information regarding behavior of the driver's face, but also various types of information that can be obtained by observing the driver, such as biological information that indicates brain waves, heart rate, or the like.

In the driver monitoring apparatus according to the above aspect, the driver state estimating unit may obtain, as the driving concentration information, attention state information that indicates an attention state of the driver and readiness information that indicates a degree of readiness for driving of the driver. According to this configuration, the state of the driver can be monitored from two viewpoints, namely the attention state of the driver and the state of readiness for driving.

In the driver monitoring apparatus according to the above aspect, the attention state information may indicate the attention state of the driver in a plurality of levels, and the readiness information may indicate the degree of readiness for driving of the driver in a plurality of level. According to this configuration, the degree of concentration of the driver on driving can be expressed in multiple levels.

The driver monitoring apparatus according to the above aspect may further include an alert unit configured to alert the driver to enter a state suited to driving the vehicle in a plurality of levels in accordance with a level of the attention state of the driver indicated by the attention state information and a level of the readiness for driving of the driver indicated by the readiness information. According to this configuration, it is possible to evaluate the state of the driver in multiple levels and give alerts that are suited to various states.

In the driver monitoring apparatus according to the above aspect, the driver state estimating unit may obtain, as the driving concentration information, action state information that indicates an action state of the driver from among a plurality of predetermined action states that are each set in correspondence with a degree of concentration of the driver on driving. According to this configuration, the degree of concentration of the driver on driving can be monitored based on action states of the driver.

In the driver monitoring apparatus according to the above aspect, the observation information obtaining unit may obtain, as the facial behavior information, information regarding at least one of whether or not the face of the driver was detected, a face position, a face orientation, a face movement, a gaze direction, a position of a facial organ, and an eye open/closed state, by performing predetermined image analysis on the captured image that was obtained. According to this configuration, the state of the driver can be estimated using information regarding at least one of whether or not the face of the driver was detected, a face position, a face orientation, a face movement, a gaze direction, a position of a facial organ, and an eye open/closed state.

The driver monitoring apparatus according to the above aspect may further include a resolution converting unit configured to lower a resolution of the obtained captured image to generate a low-resolution captured image, and the driver state estimating unit may input the low-resolution captured image to the learner. According to this configuration, the learner receives an input of not only the captured image, but also the observation information that includes the facial behavior information regarding behavior of the driver's face. For this reason, there are cases where detailed information is not needed from the captured image. In view of this, according to the above configuration, the low-resolution captured image is input to the learner. Accordingly, it is possible to reduce the amount of calculation in the computational processing performed by the learner, and it is possible to suppress the load borne by the processor when monitoring the driver. Note that even if the resolution is lowered, features regarding the posture of the driver can be extracted from the captured image. For this reason, by using the low-resolution captured image along with the observation information, it is possible to estimate the degree of concentration of the driver on driving with consideration given to various states of the driver.

In the driver monitoring apparatus according to the above aspect, the learner may include a fully connected neural network to which the observation information is input, a convolutional neural network to which the captured image is input, and a connection layer that connects output from the fully connected neural network and output from the convolutional neural network. The fully connected neural network is a neural network that has a plurality of layers that each include one or more neurons (nodes) , and the one or more neurons in each layer are connected to all of the neurons included in an adjacent layer. Also, the convolutional neural network is a neural network that includes one or more convolutional layers and one or more pooling layers, and the convolutional layers and the pooling layers are arranged alternatingly. The learner in the above configuration includes two types of neural networks on the input side, namely the fully connected neural network and the convolutional neural network. Accordingly, it is possible to perform analysis that is suited to each type of input, and it is possible to increase the accuracy of estimating the state of the driver.

In the driver monitoring apparatus according to the above aspect, the learner may further include a recurrent neural network to which output from the connection layer is input. A recurrent neural network is to a neural network having an inner loop, such as a path from an intermediate layer to an input layer.

According to this configuration, by using time series data for the observation information and the captured image, the state of the driver can be estimated with consideration given to past states. Accordingly, it is possible to increase the accuracy of estimating the state of the driver.

In the driver monitoring apparatus according to the above aspect, the recurrent neural network may include a long short-term memory (LSTM) block. The long short-term memory block includes an input gate and an output gate, and is configured to learn time points at which information is stored and output . There is also a type of long short-term memory block that includes a forget gate so as to be able to learn time points to forget information. Hereinafter, the long short-term memory block is also called an “LSTM block”. According to the above configuration, the state of the driver can be estimated with consideration give to not only short-term dependencies, but also long-term dependencies. Accordingly, it is possible to increase the accuracy of estimating the state of the driver.

In the driver monitoring apparatus according to the above aspect, the driver state estimating unit further inputs, to the learner, influential factor information regarding a factor that influences the degree of concentration of the driver on driving. According to this configuration, the influential factor information is also used when estimating the state of the driver, thus making it is possible to increase the accuracy of estimating the state of the driver. Note that the influential factor information may include various types of factors that can possibly influence the degree of concentration of the driver, such as speed information indicating the traveling speed of the vehicle, surrounding environment information indicating the situation in the surrounding environment of the vehicle (e.g., measurement results from a radar device and images captured by a camera), and weather information indicating weather.

A drive monitoring method according to an aspect of the present invention is a method in which a computer executes: an image obtaining step of obtaining a captured image from an imaging apparatus arranged so as to capture an image of a driver seated in a driver seat of a vehicle; an observation information obtaining step of obtaining observation information regarding the driver, the observation information including facial behavior information regarding behavior of a face of the driver; and an estimating step of inputting the captured image and the observation information to a trained learner that has been trained to estimate a degree of concentration of the driver on driving, and obtaining, from the learner, driving concentration information regarding the degree of concentration of the driver on driving. According to this configuration, the degree of concentration of the driver on driving can be estimated with consideration given to various states that the driver can possibly be in.

In the drive monitoring method according to the above aspect, in the estimating step, the computer may obtain, as the driving concentration information, attention state information that indicates an attention state of the driver and readiness information that indicates a degree of readiness for driving of the driver. According to this configuration, the state of the driver can be monitored from two viewpoints, namely the attention state of the driver and the state of readiness for driving.

In the drive monitoring method according to the above aspect, the attention state information may indicate the attention state of the driver in a plurality of levels, and the readiness information may indicate the degree of readiness for driving of the driver in a plurality of levels. According to this configuration, the degree of concentration of the driver on driving can be expressed in multiple levels.

In the drive monitoring method according to the above aspect, the computer may further execute an alert step of alerting the driver to enter a state suited to driving the vehicle in a plurality of levels in accordance with a level of the attention state of the driver indicated by the attention state information and a level of the readiness for driving of the driver indicated by the readiness information. According to this configuration, it is possible to evaluate the state of the driver in multiple levels and give alerts that are suited to various states.

In the drive monitoring method according to the above aspect, in the estimating step, the computer may obtain, as the driving concentration information, action state information that indicates an action state of the driver from among a plurality of predetermined action states that are each set in correspondence with a degree of concentration of the driver on driving. According to this configuration, the degree of concentration of the driver on driving can be monitored based on action states of the driver.

In the drive monitoring method according to the above aspect, in the observation information obtaining step, the computer may obtain, as the facial behavior information, information regarding at least one of whether or not the face of the driver was detected, a face position, a face orientation, a face movement, a gaze direction, a position of a facial organ, and an eye open/closed state, by performing predetermined image analysis on the captured image that was obtained in the image obtaining step. According to this configuration, the state of the driver can be estimated using information regarding at least one of whether or not the face of the driver was detected, a face position, a face orientation, a face movement, a gaze direction, a position of a facial organ, and an eye open/closed state.

In the drive monitoring method according to the above aspect, the computer may further execute a resolution converting step of lowering a resolution of the obtained captured image to generate a low-resolution captured image, and in the estimating step, the computer may input the low-resolution captured image to the learner. According to this configuration, it is possible to reduce the amount of calculation in the computational processing performed by the learner, and it is possible to suppress the load borne by the processor when monitoring the driver.

In the drive monitoring method according to the above aspect, the learner may include a fully connected neural network to which the observation information is input, a convolutional neural network to which the captured image is input, and a connection layer that connects output from the fully connected neural network and output from the convolutional neural network. According to this configuration, it is possible to perform analysis that is suited to each type of input, and it is possible to increase the accuracy of estimating the state of the driver.

In the drive monitoring method according to the above aspect, the learner may further include a recurrent neural network to which output from the connection layer is input. According to this configuration, it is possible to increase the accuracy of estimating the state of the driver.

In the drive monitoring method according to the above aspect, the recurrent neural network may include a long short-term memory (LSTM) block. According to this configuration, it is possible to increase the accuracy of estimating the state of the driver.

In the drive monitoring method according to the above aspect, in the estimating step, the computer may further input, to the learner, influential factor information regarding a factor that influences the degree of concentration of the driver on driving. According to this configuration, it is possible to increase the accuracy of estimating the state of the driver.

Also, a learning apparatus according to an aspect of the present invention includes: a training data obtaining unit configured to obtain, as training data, a set of a captured image obtained from an imaging apparatus arranged so as to capture an image of a driver seated in a driver seat of a vehicle, observation information that includes facial behavior information regarding behavior of a face of the driver, and driving concentration information regarding a degree of concentration of the driver on driving; and a learning processing unit configured to train a learner to output an output value that corresponds to the driving concentration information when the captured image and the observation information are input. According to this configuration, it is possible to construct a trained learner for use when estimating the degree of concentration of the driver on driving.

Also, a learning method according to an aspect of the present invention is a method in which a computer executes: a training data obtaining step of obtaining, as training data, a set of a captured image obtained from an imaging apparatus arranged so as to capture an image of a driver seated in a driver seat of a vehicle, observation information that includes facial behavior information regarding behavior of a face of the driver, and driving concentration information regarding a degree of concentration of the driver on driving; and a learning processing step of training a learner to output an output value that corresponds to the driving concentration information when the captured image and the observation information are input. According to this configuration, it is possible to construct a trained learner for use when estimating the degree of concentration of the driver on driving.

### Advantageous Effects of Invention

According to the present invention, it is possible to provide technology for making it possible to estimate the degree concentration of a driver on driving with consideration given to various states that the driver can possibly be in.

### BRIEF DESCRIPTION OF DRAWINGS

FIG. 1 schematically illustrates an example of a situation in which the present invention is applied.

FIG. 2 schematically illustrates an example of a hardware configuration of an automatic driving assist apparatus according to an embodiment.

FIG. 3 schematically illustrates an example of a hardware configuration of a learning apparatus according to an embodiment.

FIG. 4 schematically illustrates an example of a function configuration of the automatic driving assist apparatus according to an embodiment.

FIG. 5A schematically illustrates an example of attention state information according to an embodiment.

FIG. 5B schematically illustrates an example of readiness information according to an embodiment.

FIG. 6 schematically illustrates an example of a function configuration of a learning apparatus according to an embodiment.

FIG. 7 schematically illustrates an example of a processing procedure of the automatic driving assist apparatus according to an embodiment.

FIG. 8 schematically illustrates an example of a processing procedure of the learning apparatus according to an embodiment.

FIG. 9A schematically illustrates an example of attention state information according to a variation.

FIG. 9B schematically illustrates an example of readiness information according to a variation.

FIG. 10 schematically illustrates an example of a processing procedure of the automatic driving assist apparatus according to a variation.

FIG. 11 schematically illustrates an example of a processing procedure of the automatic driving assist apparatus according to a variation.

FIG. 12 schematically illustrates an example of a function configuration of an automatic driving assist apparatus according to a variation.

FIG. 13 schematically illustrates an example of a function configuration of an automatic driving assist apparatus according to a variation.

### DESCRIPTION OF EMBODIMENTS

An embodiment according to one aspect of the present invention (hereafter, also called the present embodiment) will be described below with reference to the drawings. Note that the embodiment described below is merely an illustrative example of the present invention in all aspects. It goes without saying that various improvements and changes can be made without departing from the scope of the present invention. More specifically, when carrying out the present invention, specific configurations that correspond to the mode of carrying out the invention may be employed as necessary. For example, the present embodiment illustrates an example in which the present invention is applied to an automatic driving assist apparatus that assists the automatic driving of an automobile. However, the present invention is not limited to being applied to a vehicle that can perform automatic driving, and the present invention may be applied to a general vehicle that cannot perform automatic driving. Note that although the data used in the present embodiment is described in natural language, such data is more specifically defined using any computer-readable language, such as a pseudo language, commands, parameters, or a machine language.

1. Application Examples

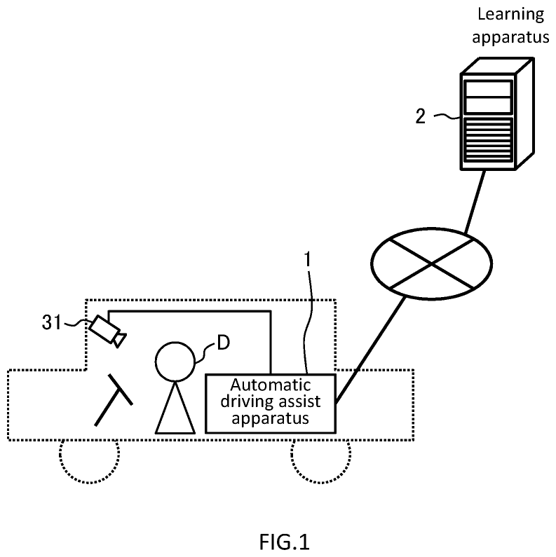

First, the following describes an example of a situation in which the present invention is applied, with reference to FIG. 1. FIG. 1 schematically illustrates an example of a situation in which an automatic driving assist apparatus 1 and a learning apparatus 2 according to the present embodiment are applied.

As shown in FIG. 1, the automatic driving assist apparatus 1 according to the present embodiment is a computer that assists the automatic driving of a vehicle while monitoring a driver D with use of a camera 31. The automatic driving assist apparatus 1 according to the present embodiment corresponds to a “driver monitoring apparatus” of the present invention.

Specifically, the automatic driving assist apparatus 1 obtains a captured image from the camera 31, which is arranged so as to capture an image of the driver D seated in the driver seat of the vehicle. The camera 31 corresponds to an “imaging apparatus” of the present invention. The automatic driving assist apparatus 1 also obtains driver observation information that includes facial behavior information regarding behavior of the face of the driver D. The automatic driving assist apparatus 1 inputs the obtained captured image and observation information to a learner (neural network 5 described later) that has been trained through machine learning to estimate the degree to which the driver is concentrating on driving, and obtains driving concentration information, which indicates the degree to which the driver D is concentrating on driving, from the learner. The automatic driving assist apparatus 1 thus estimates the state of the driver D, or more specifically, the degree to which the driver D is concentrating on driving (hereinafter, called the “degree of driving concentration”).

The learning apparatus 2 according to the present embodiment is a computer that constructs the learner that is used in the automatic driving assist apparatus 1, or more specifically, a computer that trains, through machine learning, the learner to output driver concentration information, which indicates the degree to which the driver D is concentrating on driving, in response to an input of a captured image and observation information. Specifically, the learning apparatus 2 obtains a set of captured images, observation information, and driving concentration information as training data. The captured images and the observation information are used as input data, and the driving concentration information is used as teaching data. More specifically, the learning apparatus 2 trains a learner (neural network 6 described later) to output output values corresponding to the driving concentration information in response to the input of captured images and observation information. This obtains the trained learner that is used in the automatic driving assist apparatus 1. The automatic driving assist apparatus 1 obtains the trained learner constructed by the learning apparatus 2 via a network for example. The network may be selected as appropriate from, for example, the Internet, a wireless communication network, a mobile communication network, a telephone network, and a dedicated network.

As described above, in the present embodiment, the state of the driver D is estimated using a trained learner that has been trained in order to estimate the degree to which a driver is concentrating on driving. The information that is input to the learner includes observation information, which is obtained by observing the driver and includes facial behavior information regarding behavior of the driver's face, as well as captured images obtained from the camera 31 that is arranged so as to capture images of the driver seated in the driver seat of the vehicle. For this reason, estimation is performed using not only the behavior of the face of the driver D, but also the state of the body (e.g., body orientation and posture) of the driver D that can be analyzed using the captured images. Accordingly, the present embodiment makes it possible to estimate the degree to which the driver D is concentrating on driving with consideration given to various states that the driver D can possibly be in.

2. Example Structure

Hardware Configuration

Automatic Driving Assist Apparatus

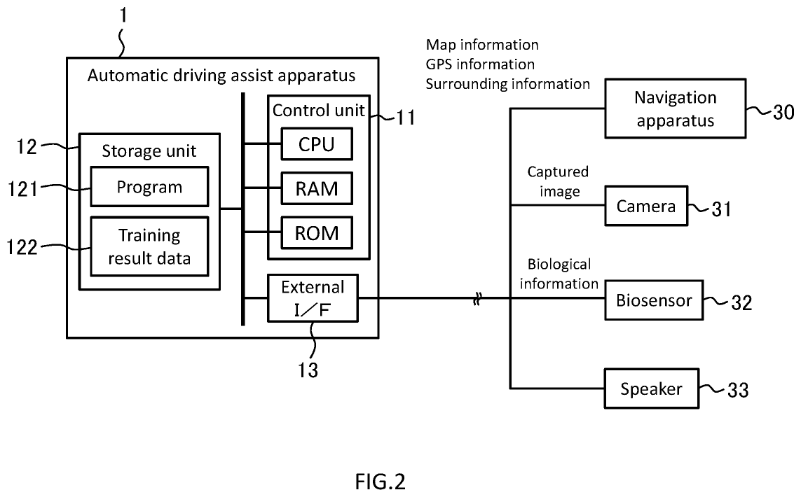

The hardware configuration of the automatic driving assist apparatus 1 according to the present embodiment will now be described with reference to FIG. 2. FIG. 2 schematically illustrates an example of the hardware configuration of the automatic driving assist apparatus 1 according to the present embodiment.

As shown in FIG. 2, the automatic driving assist apparatus 1 according to the present embodiment is a computer including a control unit 11, a storage unit 12, and an external interface 13 that are electrically connected to one another. In FIG. 2, the external interface is abbreviated as an external I/F.

The control unit 11 includes, for example, a central processing unit (CPU) as a hardware processor, a random access memory (RAM), and a read only memory (ROM), and the control unit 11 controls constituent elements in accordance with information processing. The storage unit 12 includes, for example, a RAM and a ROM, and stores a program 121, training result data 122, and other information. The storage unit 12 corresponds to a “memory”.

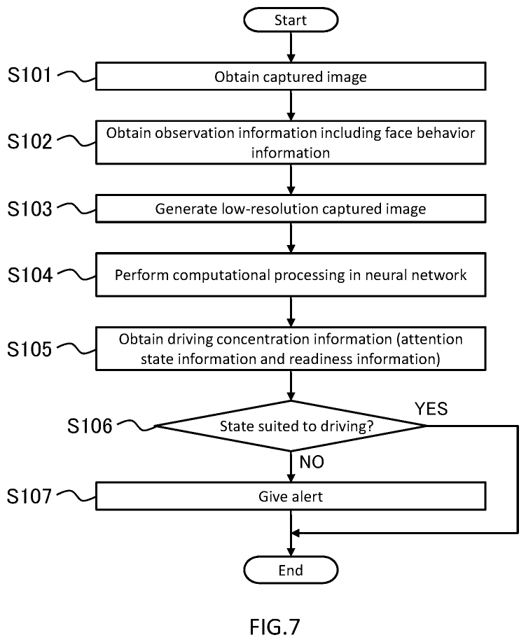

The program 121 is a program for causing the automatic driving assist apparatus 1 to implement later-described information processing (FIG. 7) for estimating the state of the driver D. The training result data 122 is used to set the trained learner. This will be described in detail later.

The external interface 13 is for connection with external devices, and is configured as appropriate depending on the external devices to which connections are made. In the present embodiment, the external interface 13 is, for example, connected to a navigation device 30, the camera 31, a biosensor 32, and a speaker 33 through a Controller Area Network (CAN).

The navigation device 30 is a computer that provides routing guidance while the vehicle is traveling. The navigation device 30 may be a known car navigation device. The navigation device 30 measures the position of the vehicle based on a global positioning system (GPS) signal, and provides routing guidance using map information and surrounding information about nearby buildings and other objects. The information indicating the position of the vehicle measured based on a GPS signal is hereafter referred to as “GPS information”.

The camera 31 is arranged so as to capture images of the driver D seated in the driver seat of the vehicle. For example, in the example in FIG. 1, the camera 31 is arranged at a position that is above and in front of the driver seat. However, the position of the camera 31 is not limited to this example, and the position may be selected as appropriate according to the implementation, as long as it is possible to capture images of the driver D seated in the driver seat. Note that the camera 31 may be a typical digital camera, video camera, or the like.

The biosensor 32 is configured to obtain biological information regarding the driver D. There are no particular limitations on the biological information that is to be obtained, and examples include brain waves and heart rate. The biosensor 32 need only be able to obtain the biological information that is required, and it is possible to use a known brain wave sensor, heart rate sensor, or the like. The biosensor 32 is attached to a body part of the driver D that corresponds to the biological information that is to be obtained.

The speaker 33 is configured to output sound. The speaker 33 is used to alert the driver D to enter a state suited to driving of the vehicle if the driver D is not in a state suited to driving the vehicle while the vehicle is traveling. This will be described in detail later.

Note that the external interface 13 may be connected to an external device other than the external devices described above. For example, the external interface 13 may be connected to a communication module for data communication via a network.

The external interface 13 is not limited to making a connection with the external devices described above, and any other external device may be selected as appropriate depending on the implementation.

In the example shown in FIG. 2, the automatic driving assist apparatus 1 includes one external interface 13. However, the external interface 13 may be separately provided for each external device to which a connection is made. The number of external interfaces 13 may be selected as appropriate depending on the implementation.

Note that in the specific hardware configuration of the automatic driving assist apparatus 1, constituent elements may be omitted, substituted, or added as appropriate depending on the implementation. For example, the control unit 11 may include multiple hardware processors. The hardware processors may be a microprocessor, an FPGA (Field-Programmable Gate Array), or the like. The storage unit 12 may be the RAM and the ROM included in the control unit 11. The storage unit 12 may also be an auxiliary storage device such as a hard disk drive or a solid state drive. The automatic driving assist apparatus 1 may be an information processing apparatus dedicated to an intended service or may be a general-purpose computer.

Learning Apparatus

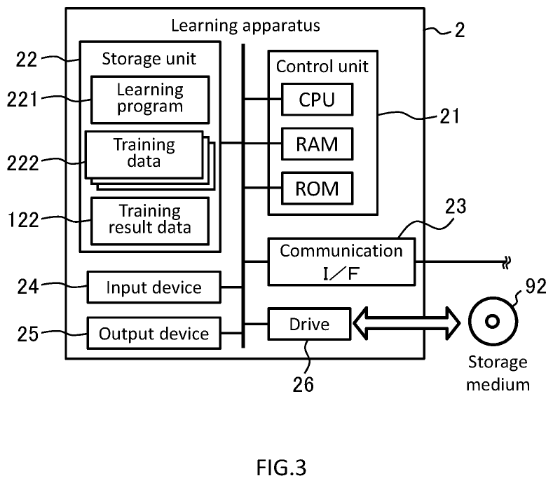

An example of the hardware configuration of the learning apparatus 2 according to the present embodiment will now be described with reference to FIG. 3. FIG. 3 schematically illustrates an example of the hardware configuration of the learning apparatus 2 according to the present embodiment.

As shown in FIG. 3, the learning apparatus 2 according to the present embodiment is a computer including a control unit 21, a storage unit 22, a communication interface 23, an input device 24, an output device 25, and a drive 26, which are electrically connected to one another. In FIG. 3, the communication interface is abbreviated as “communication I/F”.

Similarly to the control unit 11 described above, the control unit 21 includes, for example, a CPU as a hardware processor, a RAM, and a ROM, and executes various types of information processing based on programs and data. The storage unit 22 includes, for example, a hard disk drive or a solid state drive. The storage unit 22 stores, for example, a learning program 221 that is to be executed by the control unit 21, training data 222 used by the learner in learning, and the training result data 122 created by executing the learning program 221.

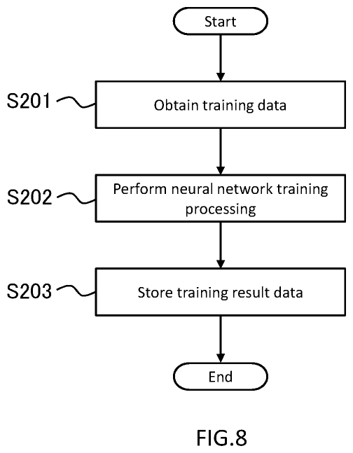

The learning program 221 is a program for causing the learning apparatus 2 to execute later-described machine learning processing (FIG. 8). The training data 222 is used to train the learner to obtain the ability to estimate the degree of driving concentration of the driver. This will be described in detail later.

The communication interface 23 is, for example, a wired local area network (LAN) module or a wireless LAN module for wired or wireless communication through a network. The learning apparatus 2 may distribute the created training data 222 to an external device via the communication interface 23.

The input device 24 is, for example, a mouse or a keyboard. The output device 25 is, for example, a display or a speaker. An operator can operate the learning apparatus 2 via the input device 24 and the output device 25.

The drive 26 is a drive device such as a compact disc (CD) drive or a digital versatile disc (DVD) drive for reading a program stored in a storage medium 92. The type of drive 26 maybe selected as appropriate depending on the type of storage medium 92. The learning program 221 and the training data 222 may be stored in the storage medium 92.

The storage medium 92 stores programs or other information in an electrical, magnetic, optical, mechanical, or chemical manner to allow a computer or another device or machine to read the recorded programs or other information. The learning apparatus 2 may obtain the learning program 221 and the training data 222 from the storage medium 92.

In FIG. 3, the storage medium 92 is a disc-type storage medium, such as a CD and a DVD. However, the storage medium 92 is not limited to being a disc, and may be a medium other than a disc. One example of the storage medium other than a disc is a semiconductor memory such as a flash memory.

Note that in the specific hardware configuration of the learning apparatus 2, constituent elements may be omitted, substituted, or added as appropriate depending on the implementation. For example, the control unit 21 may include multiple hardware processors. The hardware processors may be a microprocessor, an FPGA (Field-Programmable Gate Array), or the like. The learning apparatus 2 may include multiple information processing apparatuses. The learning apparatus 2 may also be an information processing apparatus dedicated to an intended service, or may be a general-purpose server or a personal computer (PC).

Function Configuration

Automatic Driving Assist Apparatus

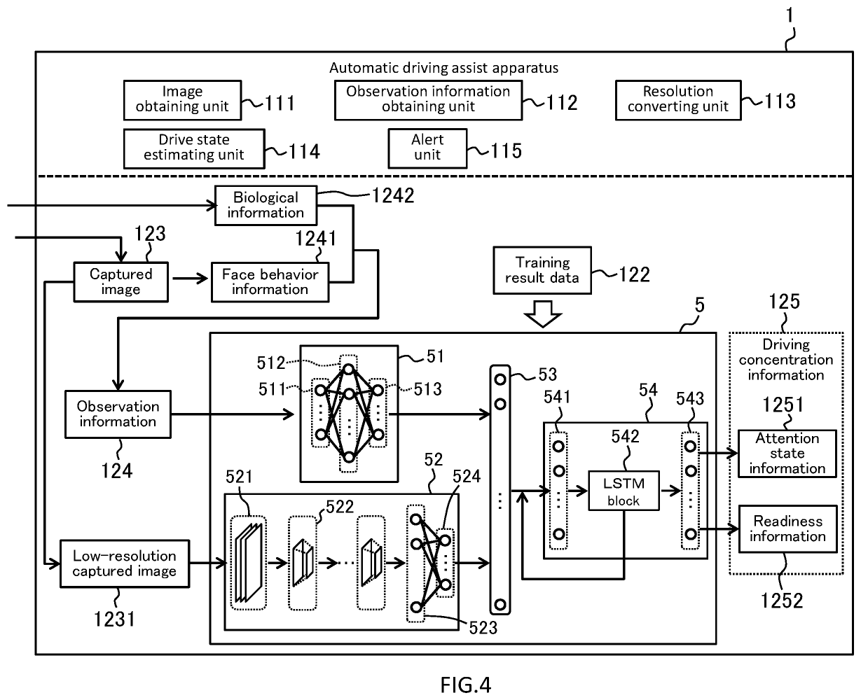

An example of the function configuration of the automatic driving assist apparatus 1 according to the present embodiment will now be described with reference to FIG. 4. FIG. 4 schematically illustrates an example of the function configuration of the automatic driving assist apparatus 1 according to the present embodiment.

The control unit 11 included in the automatic driving assist apparatus 1 loads the program 121 stored in the storage unit 12 to the RAM. The CPU in the control unit 11 then interprets and executes the program 121 loaded in the RAM to control constituent elements. The automatic driving assist apparatus 1 according to the present embodiment thus functions as a computer including an image obtaining unit 111, an observation information obtaining unit 112, a resolution converting unit 113, a drive state estimating unit 114, and an alert unit 115 as shown in FIG. 4.

The image obtaining unit 111 obtains a captured image 123 from the camera 31 that is arranged so as to capture images of the driver D seated in the driver seat of the vehicle. The observation information obtaining unit 112 obtains observation information 124 that includes facial behavior information 1241 regarding behavior of the face of the driver D and biological information 1242 obtained by the biosensor 32. In the present embodiment, the facial behavior information 1241 is obtained by performing image analysis on the captured image 123. Note that the observation information 124 is not limited to this example, and the biological information 1242 may be omitted. In this case, the biosensor 32 may be omitted.

The resolution converting unit 113 lowers the resolution of the captured image 123 obtained by the image obtaining unit 111. The resolution converting unit 113 thus generates a low-resolution captured image 1231.

The drive state estimating unit 114 inputs the low-resolution captured image 1231, which was obtained by lowering resolution of the captured image 123, and the observation information 124 to a trained learner (neural network 5) that has been trained to estimate the degree of driving concentration of the driver. The drive state estimating unit 114 thus obtains, from the learner, driving concentration information 125 regarding the degree of driving concentration of the driver D. In the present embodiment, the driving concentration information 125 obtained by the drive state estimating unit 114 includes attention state information 1251 that indicates the attention state of the driver D and readiness information 1252 that indicates the extent to which the driver D is ready to drive. The processing for lowering the resolution may be omitted. In this case, the drive state estimating unit 114 may input the captured image 123 to the learner.

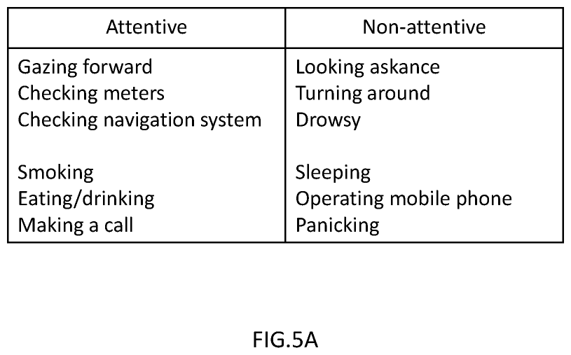

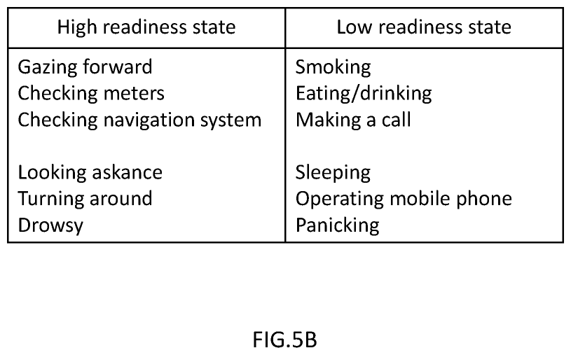

The following describes the attention state information 1251 and the readiness information 1252 with reference to FIGS. 5A and 5B. FIGS. 5A and 5B show examples of the attention state information 1251 and the readiness information 1252. As shown in FIG. 5A, the attention state information 1251 of the present embodiment indicates, using one of two levels, whether or not the driver D is giving necessary attention to driving. Also, as shown in FIG. 5B, the readiness information 1252 of the present embodiment indicates, using one of two levels, whether the driver is in a state of high readiness or low readiness for driving.

The relationship between the action state of the driver D and the attention state and readiness can be set as appropriate. For example, if the driver D is in an action state such as “gazing forward”, “checking meters”, or “checking navigation system”, it is possible to estimate that the driver D is giving necessary attention to driving and is in a state of high readiness for driving. In view of this, in the present embodiment, if the driver D is in action states such as “gazing forward”, “checking meters”, and “checking navigation system”, the attention state information 1251 is set to indicate that the driver D is giving necessary attention to driving, and the readiness information 1252 is set to indicate that the driver D is in a state of high readiness for driving. This “readiness” indicates the extent to which the driver is prepared to drive, such as the extent to which the driver D can return to manually driving the vehicle in the case where an abnormality or the like occurs in the automatic driving apparatus 1 and automatic driving can no longer be continued. Note that “gazing forward” refers to a state in which the driver D is gazing in the direction in which the vehicle is traveling. Also, “checking meters” refers to a state in which the driver D is checking a meter such as the speedometer of the vehicle. Furthermore, “checking navigation system” refers to a state in which the driver D is checking the routing guidance provided by the navigation device 30.

Also, if the driver D is in an action state such as “smoking”, “eating/drinking”, or “making a call”, it is possible to estimate that the driver D is giving necessary attention to driving, but is in a state of low readiness for driving. In view of this, in the present embodiment, if the driver D is in action states such as “gazing forward”, “checking meters”, and “checking navigation system”, the attention state information 1251 is set to indicate that the driver D is giving necessary attention to driving, and the readiness information 1252 is set to indicate that the driver D is in a state of low readiness for driving. Note that “smoking” refers to a state in which the driver D is smoking. Also, “eating/drinking” refers to a state in which the driver D is eating or drinking. Furthermore, “making a call” refers to a state in which the driver D is talking on a telephone such as a mobile phone.

Also, if the driver D is in an action state such as “looking askance”, “turning around”, or “drowsy”, the attention state information is set to indicate that the driver D is not giving necessary attention to driving, but the readiness information is set to indicate that the driver D is in a state of high readiness for driving. In view of this, in the present embodiment, if the driver D is in action states such as “looking askance”, “turning around”, or “drowsy”, the attention state information 1251 is set to indicate that the driver D is not giving necessary attention to driving, and the readiness information 1252 is set to indicate that the driver D is in a state of high readiness for driving. Note that “looking askance” refers to a state in which the driver D is not looking forward. Also, “turning around” refers to a state in which the driver D has turned around toward the back seats. Furthermore, “drowsy” refers to a state in which the driver D has become drowsy.

Also, if the driver D is in an action state such as “sleeping”, “operating mobile phone”, or “panicking”, it is possible to estimate that the driver D is not giving necessary attention to driving, and is in a state of low readiness for driving. In view of this, in the present embodiment, if the driver D is in action states such as “sleeping”, “operating mobile phone”, or “panicking”, the attention state information 1251 is set to indicate that the driver D is not giving necessary attention to driving, and the readiness information 1252 is set to indicate that the driver D is in a state of low readiness for driving. Note that “sleeping” refers to a state in which the driver D is sleeping. Also, “operating mobile phone” refers to a state in which the driver D is operating a mobile phone. Furthermore, “panicking” refers to a state in which the driver D is panicking due to a sudden change in physical condition.

The alert unit 115 determines, based on the driving concentration information 125, whether or not the driver D is in a state suited to driving the vehicle, or in other words, whether or not the degree of driving concentration of the driver D is high. Upon determining that the driver D is not in a state suited to driving the vehicle, the speaker 33 is used to give an alert for prompting the driver D to enter a state suited to driving the vehicle.

Learner

The learner will now be described. As shown in FIG. 4, the automatic driving assist apparatus 1 according to the present embodiment uses the neural network 5 as the learner trained through machine learning to estimate the degree of driving concentration of the driver. The neural network 5 according to the present embodiment is constituted by a combination of multiple types of neural networks.

Specifically, the neural network 5 is divided into four parts, namely a fully connected neural network 51, a convolutional neural network 52, a connection layer 53, and an LSTM network 54. The fully connected neural network 51 and the convolutional neural network 52 are arranged in parallel on the input side, the fully connected neural network 51 receives an input of the observation information 124, and the convolutional neural network 52 receives an input of the low-resolution captured image 1231. The connection layer 53 connects the output from the fully connected neural network 51 and the output from the convolutional neural network 52. The LSTM network 54 receives output from the connection layer 53, and outputs the attention state information 1251 and the readiness information 1252.

(a) Fully Connected Neural Network

The fully connected neural network 51 is a so-called multilayer neural network, which includes an input layer 511, an intermediate layer (hidden layer) 512, and an output layer 513 in the stated order from the input side. The number of layers included in the fully connected neural network 51 is not limited to the above example, and may be selected as appropriate depending on the implementation.

Each of the layers 511 to 513 includes one or more neurons (nodes) . The number of neurons included in each of the layers 511 to 513 may be determined as appropriate depending on the implementation. Each neuron included in each of the layers 511 to 513 is connected to all the neurons included in the adjacent layers to construct the fully connected neural network 51. Each connection has a weight (connection weight) set as appropriate.

(b) Convolutional Neural Network

The convolutional neural network 52 is a feedforward neural network with convolutional layers 521 and pooling layers 522 that are alternately stacked and connected to one another. In the convolutional neural network 52 according to the present embodiment, the convolutional layers 521 and the pooling layers 522 are alternatingly arranged on the input side. Output from the pooling layer 522 nearest the output side is input to a fully connected layer 523, and output from the fully connected layer 523 is input to an output layer 524.

The convolutional layers 521 perform convolution computations for images. Image convolution corresponds to processing for calculating a correlation between an image and a predetermined filter. An input image undergoes image convolution that detects, for example, a grayscale pattern similar to the grayscale pattern of the filter.

The pooling layers 522 perform pooling processing. In the pooling processing, image information at positions highly responsive to the filter is partially discarded to achieve invariable response to slight positional changes of the features appearing in the image.

The fully connected layer 523 connects all neurons in adjacent layers. More specifically, each neuron included in the fully connected layer 523 is connected to all neurons in the adjacent layers. The convolutional neural network 52 may include two or more fully connected layers 523. The number of neurons included in the fully connected layer 423 may be determined as appropriate depending on the implementation.

The output layer 524 is arranged nearest the output side of the convolutional neural network 52. The number of neurons included in the output layer 524 may be determined as appropriate depending on the implementation. Note that the structure of the convolutional neural network 52 is not limited to the above example, and may be set as appropriate depending on the implementation.

(c) Connection Layer

The connection layer 53 is arranged between the fully connected neural network 51 and the LSTM network 54 as well as between the convolutional neural network 52 and the LSTM network 54. The connection layer 53 connects the output from the output layer 513 in the fully connected neural network 51 and the output from the output layer 524 in the convolutional neural network 52. The number of neurons included in the connection layer 53 may be determined as appropriate depending on the number of outputs from the fully connected neural network 51 and the convolutional neural network 52.

(d) LSTM Network

The LSTM network 54 is a recurrent neural network including an LSTM block 542. A recurrent neural network is to a neural network having an inner loop, such as a path from an intermediate layer to an input layer. The LSTM network 54 has a typical recurrent neural network architecture with the intermediate layer replaced by the LSTM block 542.

In the present embodiment, the LSTM network 54 includes an input layer 541, the LSTM block 542, and an output layer 543 in the stated order from the input side, and the LSTM network 54 has a path for returning from the LSTM block 542 to the input layer 541, as well as a feedforward path. The number of neurons included in each of the input layer 541 and the output layer 543 may be determined as appropriate depending on the implementation.

The LSTM block 542 includes an input gate and an output gate to learn time points at which information is stored and output (S. Hochreiter and J. Schmidhuber, “Long short-term memory” Neural Computation, 9(8):1735-1780, Nov. 15, 1997). The LSTM block 542 may also include a forget gate to adjust time points to forget information (Felix A. Gers, Jurgen Schmidhuber and Fred Cummins, “Learning to Forget: Continual Prediction with LSTM” Neural Computation, pages 2451-2471, October 2000). The structure of the LSTM network 54 may be set as appropriate depending on the implementation.

(e) Summary

Each neuron has a threshold, and the output of each neuron is basically determined depending on whether the sum of its inputs multiplied by the corresponding weights exceeds the threshold. The automatic driving assist apparatus 1 inputs the observation information 124 to the fully connected neural network 51, and inputs the low-resolution captured image 1231 to the convolutional neural network 52. The automatic driving assist apparatus 1 then determines whether neurons in the layers have fired, starting from the layer nearest the input side. The automatic driving assist apparatus 1 thus obtains output values corresponding to the attention state information 1251 and the readiness information 1252 from the output layer 543 of the neural network 5.

Note that the training result data 122 includes information indicating the configuration of the neural network 5 (e.g., the number of layers in each network, the number of neurons in each layer, the connections between neurons, and the transfer function of each neuron), the connection weights between neurons, and the threshold of each neuron. The automatic driving assist apparatus 1 references the training result data 122 and sets the trained neural network 5 that is to be used in processing for estimating the degree of driving concentration of the driver D.

Learning Apparatus

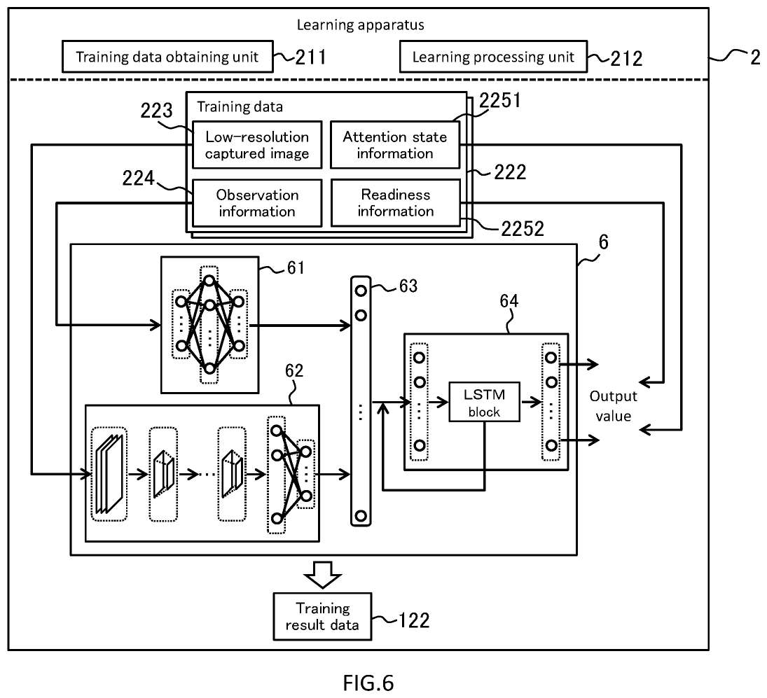

An example of the function configuration of the learning apparatus 2 according to the present embodiment will now be described with reference to FIG. 6. FIG. 6 schematically illustrates an example of the function configuration of the learning apparatus 2 according to the present embodiment.

The control unit 21 included in the learning apparatus 2 loads the learning program 221 stored in the storage unit 22 to the RAM. The CPU in the control unit 21 then interprets and executes the learning program 221 loaded in the RAM to control constituent elements. The learning apparatus 2 according to the present embodiment thus functions as a computer that includes a training data obtaining unit 211 and a learning processing unit 212 as shown in FIG. 6.

The training data obtaining unit 211 obtains a captured image captured by an imaging apparatus installed to capture an image of the driver seated in the driver seat of the vehicle, driver observation information that includes facial behavior information regarding behavior of the driver's face, and driving concentration information regarding the degree to which the driver is concentrating on driving, as a set of training data. The captured image and the observation information are used as input data. The driving concentration information is used as teaching data. In the present embodiment, the training data 222 obtained by the training data obtaining unit 211 is a set of a low-resolution captured image 223, observation information 224, attention state information 2251, and readiness information 2252. The low-resolution captured image 223 and the observation information 224 correspond to the low-resolution captured image 1231 and the observation information 124 that were described above. The attention state information 2251 and the readiness information 2252 correspond to the attention state information 1251 and the readiness information 1252 of the driving concentration information 125 that were described above. The learning processing unit 212 trains the learner to output output values that correspond to the attention state information 2251 and the readiness information 2252 when the low-resolution captured image 223 and the observation information 224 are input.

As shown in FIG. 6, the learner to be trained in the present embodiment is a neural network 6. Similarly to the neural network 5, the neural network 6 includes a fully connected neural network 61, a convolutional neural network 62, a connection layer 63, and an LSTM network 64. The fully connected neural network 61, the convolutional neural network 62, the connection layer 63, and the LSTM network 64 are respectively similar to the fully connected neural network 51, the convolutional neural network 52, the connection layer 53, and the LSTM network 54 that were described above. Through neural network training processing, the learning processing unit 212 constructs the neural network 6 such that when the observation information 224 is input to the fully connected neural network 61 and the low-resolution captured image 223 is input to the convolutional neural network 62, output values that correspond to the attention state information 2251 and the readiness information 2252 are output from the LSTM network 64. The learning processing unit 212 stores information items indicating the structure of the constructed neural network 6, the connection weights between neurons, and the threshold of each neuron in the storage unit 22 as the training result data 122.

Other Remarks

The functions of the automatic driving assist apparatus 1 and the learning apparatus 2 will be described in detail in the operation examples below. Note that in the present embodiment, the functions of the automatic driving assist apparatus 1 and the learning apparatus 2 are all realized by a general-purpose CPU. However, some or all of the functions may be realized by one or more dedicated processors. In the function configurations of the automatic driving assist apparatus 1 and the learning apparatus 2, functions may be omitted, substituted, or added as appropriate depending on the implementation.

3. Operation Examples

Automatic Driving Assist Apparatus

Operation examples of the automatic driving assist apparatus 1 will now be described with reference to FIG. 7. FIG. 7 is a flowchart of a procedure performed by the automatic driving assist apparatus 1. The processing procedure for estimating the state of the driver D described below corresponds to a “driver monitoring method” of the present invention. However, the processing procedure described below is merely one example, and the processing steps may be modified in any possible manner. In the processing procedure described below, steps may be omitted, substituted, or added as appropriate depending on the implementation.

Activation

The driver D first turns on the ignition power supply of the vehicle to activate the automatic driving assist apparatus 1, thus causing the activated automatic driving assist apparatus 1 to execute the program 121. The control unit 11 of the automatic driving assist apparatus 1 obtains map information, surrounding information, and GPS information from the navigation device 30, and starts automatic driving of the vehicle based on the obtained map information, surrounding information, and GPS information. Automatic driving may be controlled by a known control method. After starting automatic driving of the vehicle, the control unit 11 monitors the state of the driver D in accordance with the processing procedure described below. Note that the program execution is not limited to being triggered by turning on the ignition power supply of the vehicle, and the trigger may be selected as appropriate depending on the implementation. For example, if the vehicle includes a manual driving mode and an automatic driving mode, the program execution may be triggered by a transition to the automatic driving mode. Note that the transition to the automatic driving mode may be made in accordance with an instruction from the driver.

Step S101

In step S101, the control unit 11 operates as the image obtaining unit 111 and obtains the captured image 123 from the camera 31 arranged so as to capture an image of the driver D seated in the driver seat of the vehicle. The obtained captured image 123 may be a moving image or a still image. After obtaining the captured image 123, the control unit 11 advances the processing to step S102.

Step S102

In step S102, the control unit 11 functions as the observation information obtaining unit 112 and obtains the observation information 124 that includes the biological information 1242 and the facial behavior information 1241 regarding behavior of the face of the driver D. After obtaining the observation information 124, the control unit 11 advances the processing to step S103.

The facial behavior information 1241 may be obtained as appropriate. For example, by performing predetermined image analysis on the captured image 123 that was obtained in step S101, the control unit 11 can obtain, as the facial behavior information 1241, information regarding at least one of whether or not the face of the driver D was detected, a face position, a face orientation, a face movement, a gaze direction, a facial organ position, and an eye open/closed state.

As one example of a method for obtaining the facial behavior information 1241, first, the control unit 11 detects the face of the driver D in the captured image 123, and specifies the position of the detected face. The control unit 11 can thus obtain information regarding whether or not a face was detected and the position of the face. By continuously performing face detection, the control unit 11 can obtain information regarding movement of the face. The control unit 11 then detects organs included in the face of the driver D (eyes, mouth, nose, ears, etc.) in the detected face image. The control unit 11 can thus obtain information regarding the positions of facial organs. By analyzing the states of the detected organs (eyes, mouth, nose, ears, etc.), the control unit 11 can obtain information regarding the orientation of the face, the gaze direction, and the open/closed state of the eyes. Face detection, organ detection, and organ state analysis may be performed using known image analysis methods.

If the obtained captured image 123 is a moving image or a group of still images that are in a time series, the control unit 11 can obtain various types of information corresponding to the time series by executing the aforementioned types of image analysis on each frame of the captured image 123. The control unit 11 can thus obtain various types of information expressed by a histogram or statistical amounts (average value, variance value, etc.) as time series data.

The control unit 11 may also obtain the biological information (e.g., brain waves or heart rate) 1242 from the biosensor 32. For example, the biological information 1242 may be expressed by a histogram or statistical amounts (average value, variance value, etc.). Similarly to the facial behavior information 1241, the control unit 11 can obtain the biological information 1242 as time series data by continuously accessing the biosensor 32.

Step S103

In step S103, the control unit 11 functions as the resolution converting unit 113 and lowers the resolution of the captured image 123 obtained in step S101. The control unit 11 thus generates the low-resolution captured image 1231. The resolution may be lowered with any technique selected as appropriate depending on the implementation. For example, the control unit 11 may use a nearest neighbor algorithm, bilinear interpolation, or bicubic interpolation to generate the low-resolution captured image 1231. After generating the low-resolution captured image 1231, the control unit 11 advances the processing to step S104. Note that step S103 may be omitted.

Steps S104 and S105

In step S104, the control unit 11 functions as the drive state estimating unit 114 and executes computational processing in the neural network 5 using the obtained observation information 124 and low-resolution captured image 1231 as input for the neural network 5. Accordingly, in step S105, the control unit 11 obtains output values corresponding to the attention state information 1251 and the readiness information 1252 of the driving concentration information 125 from the neural network 5.

Specifically, the control unit 11 inputs the observation information 124 obtained in step S102 to the input layer 511 of the fully connected neural network 51, and inputs the low-resolution captured image 1231 obtained in step S103 to the convolutional layer 521 arranged nearest the input side in the convolutional neural network 52. The control unit 11 then determines whether each neuron in each layer fires, starting from the layer nearest the input side. The control unit 11 thus obtains output values corresponding to the attention state information 1251 and the readiness information 1252 from the output layer 543 of the LSTM network 54.

Steps S106 and S107

In step S106, the control unit 11 functions as the alert unit 115 and determines whether or not the driver D is in a state suited to driving the vehicle, based on the attention state information 1251 and the readiness information 1252 that were obtained in step S105. Upon determining that the driver D is in a state suited to driving the vehicle, the control unit 11 skips the subsequent step S107 and ends processing pertaining to this operation example. However, upon determining that the driver D is not in a state suited to driving the vehicle, the control unit 11 executes the processing of the subsequent step S107. Specifically, the control unit 11 uses the speaker 33 to give an alert to prompt the driver D to enter a state suited to driving the vehicle, and then ends processing pertaining to this operation example.

The criteria for determining whether or not the driver D is in a state suited to driving the vehicle may be set as appropriate depending on the implementation. For example, a configuration is possible in which, in the case where the attention state information 1251 indicates that the driver D is not giving necessary attention to driving, or the readiness information 1252 indicates that the driver D is in a low state of readiness for driving, the control unit 11 determines that the driver D is not in a state suited to driving the vehicle, and gives the alert in step S107. Also, in the case where the attention state information 1251 indicates that the driver D is not giving necessary attention to driving, and the readiness information 1252 indicates that the driver D is in a low state of readiness for driving, the control unit 11 may determine that the driver D is not in a state suited to driving the vehicle, and give the alert in step S107.

Furthermore, in the present embodiment, the attention state information 1251 indicates, using one of two levels, whether or not the driver D is giving necessary attention to driving, and the readiness information 1252 indicates, using one of two levels, whether the driver is in a state of high readiness or low readiness for driving. For this reason, the control unit 11 may give different levels of alerts depending on the level of the attention of the driver D indicated by the attention state information 1251 and the level of the readiness of the driver D indicated by the readiness information 1252.

For example, in the case where the attention state information 1251 indicates that the driver D is not giving necessary attention to driving, the control unit 11 may output, as an alert from the speaker 33, audio for prompting the driver D to give necessary attention to driving. Also, in the case where the readiness information 1252 indicates that the driver D is in a state of low readiness for driving, the control unit 11 may output, as an alert from the speaker 33, audio for prompting the driver D to increase their readiness for driving. Furthermore, in the case where the attention state information 1251 indicates that the driver D is not giving necessary attention to driving, and the readiness information 1252 indicates that the driver D is in a state of low readiness for driving, the control unit 11 may give a more forceful alert than in the above two cases (e.g., may increase the volume or emit a beeping noise).

As described above, the automatic driving assist apparatus 1 monitors the degree of driving concentration of the driver D during the automatic driving of the vehicle. Note that the automatic driving assist apparatus 1 may continuously monitor the degree of driving concentration of the driver D by repeatedly executing the processing of steps S101 to S107. Also, while repeatedly executing the processing of steps S101 to S107, the automatic driving assist apparatus 1 may stop the automatic driving if it has been determined multiple successive times in step S106 that the driver D is not in a state suited to driving the vehicle. In this case, for example, after determining multiple successive times that the driver D is not in a state suited to driving the vehicle, the control unit 11 may set a stopping section for safely stopping the vehicle by referencing the map information, surrounding information, and GPS information. The control unit 11 may then output an alert to inform the driver D that the vehicle is to be stopped, and may automatically stop the vehicle in the set stopping section. The vehicle can thus be stopped if the degree of driving concentration of the driver D is continuously in a low state.

Learning Apparatus

An operation example of the learning apparatus 2 will now be described with reference to FIG. 8. FIG. 8 is a flowchart illustrating an example a processing procedure performed by the learning apparatus 2. Note that the processing procedure described below associated with machine learning by the learner is an example of the “learning method” of the present invention. However, the processing procedure described below is merely one example, and the processing steps may be modified in any possible manner. In the processing procedure described below, steps may be omitted, substituted, or added as appropriate depending on the implementation.

Step S201

In step S201, the control unit 21 of the learning apparatus 2 functions as the training data obtaining unit 211 and obtains, as the training data 222, a set of the low-resolution captured image 223, the observation information 224, the attention state information 2251, and the readiness information 2252.

The training data 222 is used to train the neural network 6 through machine learning to estimate the degree of driving concentration of the driver. The training data 222 described above is generated by, for example, preparing a vehicle with the camera 31, capturing images of the driver seated in the driver seat in various states, and associating each captured image with the corresponding imaged states (attention states and degrees of readiness). The low-resolution captured image 223 can be obtained by performing the same processing as in step S103 described above on the captured images. Also, the observation information 224 can be obtained by performing the same processing as in step S102 described above on the captured images. Furthermore, the attention state information 2251 and the readiness information 2252 can be obtained by receiving an input of the states of the driver appearing in the captured images as appropriate.

Note that the training data 222 may be generated manually by an operator through the input device 24 or may be generated automatically by a program. The training data 222 may be collected from an operating vehicle at appropriate times. The training data 222 maybe generated by any information processing apparatus other than the learning apparatus 2. When the training data 222 is generated by the learning apparatus 2, the control unit 21 may obtain the training data 222 by performing the process of generating the training data 222 in step S201. When the training data 222 is generated by an information processing apparatus other than the learning apparatus 2, the learning apparatus 2 may obtain the training data 222 generated by the other information processing apparatus through, for example, a network or the storage medium 92. Furthermore, the number of sets of training data 222 obtained in step S201 may be determined as appropriate depending on the implementation to train the neural network 6 through learning.

Step S202

In step S202, the control unit 21 functions as the learning processing unit 212 and trains, using the training data 222 obtained in step S201, the neural network 6 through machine learning to output output values corresponding to the attention state information 2251 and the readiness information 2252 in response to an input of the low-resolution captured image 223 and the observation information 224.

More specifically, the control unit 21 first prepares the neural network 6 that is to be trained. The architecture of the neural network 6 that is to be prepared, the default values of the connection weights between the neurons, and the default threshold of each neuron may be provided in the form of a template or may be input by an operator. For retraining, the control unit 21 may prepare the neural network 6 based on the training result data 122 to be relearned.

Next, the control unit 21 trains the neural network 6 using the low-resolution captured image 223 and the observation information 224, which are included in the training data 222 that was obtained in step S201, as input data, and using the attention state information 2251 and the readiness information 2252 as teaching data. The neural network 6 may be trained by, for example, a stochastic gradient descent method.

For example, the control unit 21 inputs the observation information 224 to the input layer of the fully connected neural network 61, and inputs the low-resolution captured image 223 to the convolutional layer nearest the input side of the convolutional neural network 62. The control unit 21 then determines whether each neuron in each layer fires, starting from the layer nearest the input end. The control unit 21 thus obtains an output value from the output layer in the LSTM network 64. The control unit 21 then calculates an error between the output values obtained from the output layer in the LSTM network and the values corresponding to the attention state information 2251 and the readiness information 2252. Subsequently, the control unit 21 calculates errors in the connection weights between neurons and errors in the thresholds of the neurons using the calculated error in the output value with a backpropagation through time method. The control unit 21 then updates the connection weights between the neurons and also the thresholds of the neurons based on the calculated errors.

The control unit 21 repeats the above procedure for each set of training data 222 until the output values from the neural network 6 match the values corresponding to the attention state information 2251 and the readiness information 2252. The control unit 21 thus constructs the neural network 6 that outputs output values that correspond to the attention state information 2251 and the readiness information 2252 when the low-resolution captured image 223 and the observation information 224 are input.

Step S203

In step S203, the control unit 21 functions as the learning processing unit 212 and stores the information items indicating the structure of the constructed neural network 6, the connection weights between the neurons, and the threshold of each neuron to the storage unit 22 as training result data 122.

The control unit 21 then ends the learning process of the neural network 6 associated with this operation example.

Note that the control unit 21 may transfer the generated training result data 122 to the automatic driving assist apparatus 1 after the processing in step S203 is complete. The control unit 21 may periodically perform the learning process in steps S201 to S203 to periodically update the training result data 122. The control unit 21 may transfer the generated training result data 122 to the automatic driving assist apparatus 1 after completing every learning process and may periodically update the training result data 122 held by the automatic driving assist apparatus 1. The control unit 21 may store the generated training result data 122 to a data server, such as a network attached storage (NAS). In this case, the automatic driving assist apparatus 1 may obtain the training result data 122 from the data server.

Advantages and Effects

As described above, the automatic driving assist apparatus 1 according to the present embodiment obtains, through the processing in steps S101 to S103, the observation information 124 that includes the facial behavior information 1241 regarding the driver D and the captured image (low-resolution captured image 1231) that is obtained by the camera 31 arranged so as to capture the image of the driver D seated in the driver seat of the vehicle. The automatic driving assist apparatus 1 then inputs, in steps S104 and S105, the obtained observation information 124 and low-resolution captured image 1231 to the trained neural network (neural network 5) to estimate the degree of driving concentration of the driver D. The trained neural network is created by the learning apparatus 2 with use of training data that includes the low-resolution captured image 223, the observation information 224, the attention state information 2251, and the readiness information 2252. Accordingly, in the present embodiment, in the process of estimating the degree of driving concentration of the driver, consideration can be given to not only the behavior of the face of the driver D, but also states of the body of the driver D (e.g., body orientation and posture) that can be identified based on the low-resolution captured image. Therefore, according to the present embodiment, the degree of driving concentration of the driver D can be estimated with consideration given to various states that the driver D can possibly be in.

Also, in the present embodiment, the attention state information 1251 and the readiness information 1252 are obtained as the driving concentration information in step S105. For this reason, according to the present embodiment, it is possible to monitor the degree of driving concentration of the driver D from two viewpoints, namely the attention state of the driver D and the degree of readiness for driving. Additionally, according to the present embodiment, it is possible to give alerts from these two viewpoints in step S107.

Also, in the present embodiment, the observation information (124, 224) that includes the driver facial behavior information is used as input for the neural network (5, 6). For this reason, the captured image that is given as input to the neural network (5, 6) does not need to have a resolution high enough to identify behavior of the driver's face. In view of this, in the present embodiment, it is possible to use the low-resolution captured image (1231, 223), which is generated by lowering the resolution of the captured image obtained from the camera 31, as an input to the neural network (5, 6). This reduces the computation in the neural network (5, 6) and the load on the processor. Note that it is preferable that the low-resolution captured image (1231, 223) has a resolution that enables extraction of features regarding the posture of the driver but does not enable identifying behavior of the driver's face.

Also, the neural network 5 according to the present embodiment includes the fully connected neural network 51 and the convolutional neural network 52 at the input side. In step S104, the observation information 124 is input to the fully connected neural network 51, and the low-resolution captured image 1231 is input to the convolutional neural network 52. This makes it possible to perform analysis that is suited to each type of input. The neural network 5 according to the present embodiment also includes the LSTM network 54. Accordingly, by using time series data for the observation information 124 and the low-resolution captured image 1231, it is possible to estimate the degree of driving concentration of the driver D with consideration given to short-term dependencies as well as long-term dependencies. Thus, according to the present embodiment, it is possible to increase the accuracy of estimating the degree of driving concentration of the driver D.

4. Variations

The embodiments of the present invention described in detail above are mere examples in all respects. It goes without saying that various improvements and changes can be made without departing from the scope of the present invention. For example, the embodiments may be modified in the following forms. The same components as those in the above embodiments are hereafter given the same numerals, and the operations that are the same as those in the above embodiments will not be described. The modifications described below may be combined as appropriate.

<4.1>

The above embodiment illustrates an example of applying the present invention to a vehicle that can perform automatic driving. However, the present invention is not limited to being applied to a vehicle that can perform automatic driving, and the present invention may be applied to a vehicle that cannot perform automatic driving.

<4.2>

In the above embodiment, the attention state information 1251 indicates, using one of two levels, whether or not the driver D is giving necessary attention to driving, and the readiness information 1252 indicates, using one of two levels, whether the driver is in a state of high readiness or low readiness for driving. However, the expressions of the attention state information 1251 and the readiness information 1252 are not limited to these examples, and the attention state information 1251 may indicate, using three or more levels, whether or not the driver D is giving necessary attention to driving, and the readiness information 1252 may indicate, using three or more levels, whether the driver is in a state of high readiness or low readiness for driving.

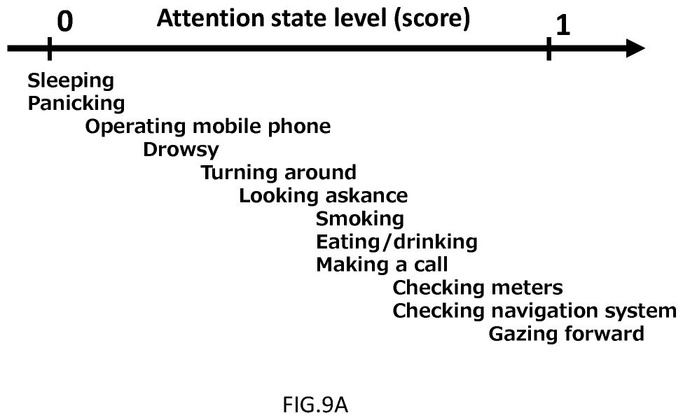

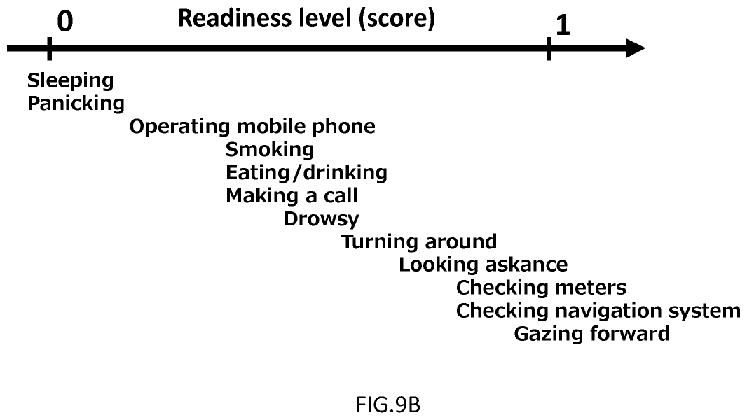

FIGS. 9A and 9B show examples of the attention state information and the readiness information according to the present variation. As shown in FIG. 9A, the attention state information according to the present variation is defined by score values from 0 to 1 that indicate the extent of attention in various action states. For example, in the example in FIG. 9A, the score value “0” is assigned for “sleeping” and “panicking”, the score value “1” is assigned for “gazing forward”, and score values between 0 and 1 are assigned for the other action states.

Similarly, the readiness information according to the present variation is defined by score values from 0 to 1 that indicate the extent of readiness relative to various action states. For example, in the example in FIG. 9B, the score value “0” is assigned for “sleeping” and “panicking”, the score value “1” is assigned for “gazing forward”, and score values between 0 and 1 are assigned for the other action states.

In this way, by assigning three or more score values for various action states, the attention state information 1251 may indicate, using three or more levels, whether or not the driver D is giving necessary attention to driving, and the readiness information 1252 may indicate, using three or more levels, whether the driver is in a state of high readiness or low readiness for driving.

In this case, in step S106, the control unit 11 may determine whether or not the driver D is in a state suited to driving the vehicle based on the score values of the attention state information and the readiness information. For example, the control unit 11 may determine whether or not the driver D is in a state suited to driving the vehicle based on whether or not the score value of the attention state information is higher than a predetermined threshold. Also, for example, the control unit 11 may determine whether or not the driver D is in a state suited to driving the vehicle based on whether or not the score value of the readiness information is higher than a predetermined threshold. Furthermore, for example, the control unit 11 may determine whether or not the driver D is in a state suited to driving the vehicle based on whether or not the total value of the score value of the attention state information and the score value of the readiness information is higher than a predetermined threshold. This threshold may be set as appropriate. Also, the control unit 11 may change the content of the alert in accordance with the score value. The control unit 11 may thus give different levels of alerts. Note that in this case where the attention state information and the readiness information are expressed by score values, the upper limit value and the lower limit value of the score values maybe set as appropriate depending on the implementation. The upper limit value of the score value is not limited to being “1”, and the lower limit value is not limited to being “0”.

<4.3>

In the above embodiment, in step S106, the degree of driving concentration of the driver D is determined using the attention state information 1251 and the readiness information 1252 in parallel. However, when determining whether or not the driver D is in a state suited to driving the vehicle, priority may be given to either the attention state information 1251 or the readiness information 1252.

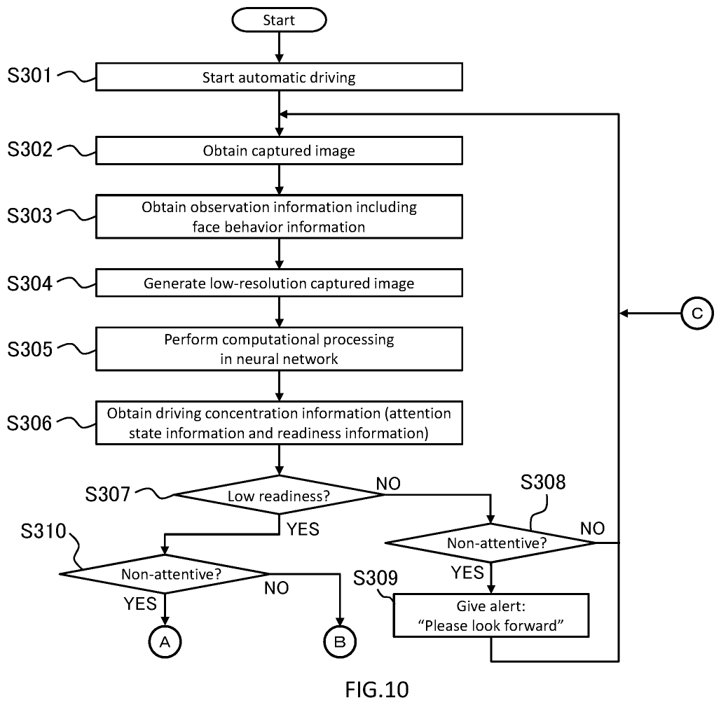

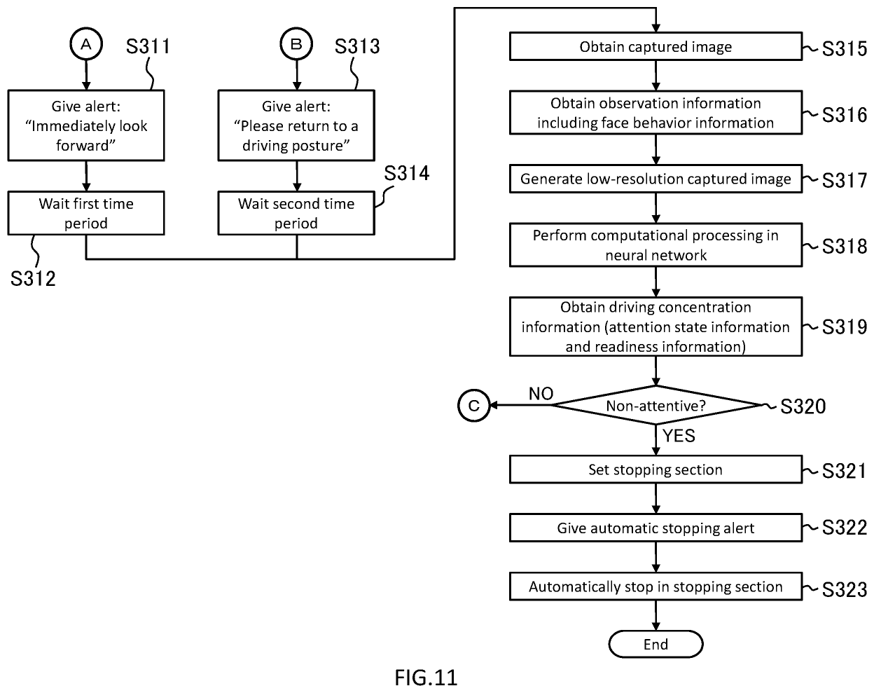

FIGS. 10 and 11 show a variation of the processing procedure described above. By carrying out the processing procedure according to the present variation, the automatic driving assist apparatus 1 ensures that at least the driver D is giving necessary attention to driving when controlling the automatic driving of the vehicle. Specifically, the automatic driving assist apparatus 1 controls the automatic driving of the vehicle as described below.

Step S301

In step S301, the control unit 11 starts the automatic driving of the vehicle. For example, similarly to the above embodiment, the control unit 11 obtains map information, surrounding information, and GPS information from the navigation device 30, and implements automatic driving of the vehicle based on the obtained map information, surrounding information, and GPS information. After starting the automatic driving of the vehicle, the control unit 11 advances the processing to step S302.

Steps S302 to S306

Steps S302 to S306 are similar to steps S101 to S105 described above. In other words, as a result of the processing in steps S302 to S306, the control unit 11 obtains the attention state information 1251 and the readiness information 1252 from the neural network 5. Upon obtaining the attention state information 1251 and the readiness information 1252, the control unit 11 advances the processing to step S307.

Step S307

In step S307, the control unit 11 determines whether or not the driver D is in a state of low readiness for driving based on the readiness information 1252 obtained in step S306. If the readiness information 1252 indicates that the driver D is in a state of low readiness for driving, the control unit 11 advances the processing to step S310. However, if the readiness information 1252 indicates that the driver D is in a state of high readiness for driving, the control unit 11 advances the processing to step S308.

Step S308

In step S308, the control unit 11 determines whether or not the driver D is giving necessary attention to driving based on the attention state information 1251 obtained in step S306. If the attention state information 1251 indicates that the driver D is not giving necessary attention to driving, the driver D is in a state of high readiness for driving, but is in a state of not giving necessary attention to driving. In this case, the control unit 11 advances the processing to step S309.

However, if the attention state information 1251 indicates that the driver D is giving necessary attention to driving, the driver D is in a state of high readiness for driving, and is in a state of giving necessary attention to driving. In this case, the control unit 11 returns the processing to step S302 and continues to monitor the driver D while performing the automatic driving of the vehicle.

Step S309

In step S309, the control unit 11 functions as the alert unit 115, and if it was determined that the driver D is in a state of high readiness for driving, but is in a state of not giving necessary attention to driving, the control unit 11 outputs, as an alert from the speaker 33, the audio “Please look forward”. The control unit 11 thus prompts the driver D to give necessary attention to driving. When this alert is complete, the control unit 11 returns the processing to step S302. Accordingly, the control unit 11 continues to monitor the driver D while performing the automatic driving of the vehicle.

Step S310

In step S310, the control unit 11 determines whether or not the driver D is giving necessary attention to driving based on the attention state information 1251 obtained in step S306. If the attention state information 1251 indicates that the driver D is not giving necessary attention to driving, the driver D is in a state of low readiness for driving, and is in a state of not giving necessary attention to driving. In this case, the control unit 11 advances the processing to step S311.

However, if the attention state information 1251 indicates that the driver D is giving necessary attention to driving, the driver D is in a state of low readiness for driving, but is in a state of giving necessary attention to driving. In this case, the control unit 11 advances the processing to step S313.

Steps S311 and S312

In step S311, the control unit 11 functions as the alert unit 115, and if it was determined that the driver D is in a state of low readiness for driving, and is in a state of not giving necessary attention to driving, the control unit 11 outputs, as an alert from the speaker 33, the audio “Immediately look forward”. The control unit 11 thus prompts the driver D to at least give necessary attention to driving. After the alert is given, in step S312, the control unit 11 waits for a first time period. After waiting for the first time period, the control unit 11 advances the processing to step S315. Note that the specific value of the first time period may be set as appropriate depending on the implementation.

Steps S313 and S314

In step S313, the control unit 11 functions as the alert unit 115, and if it was determined that the driver D is in a state of low readiness for driving, but is in a state of giving necessary attention to driving, the control unit 11 outputs, as an alert from the speaker 33, the audio “Please return to a driving posture” . The control unit 11 thus prompts the driver D to enter a state of high readiness for driving. After the alert is given, in step S314, the control unit 11 waits for a second time period that is longer than the first time period. Step S312 is executed if it is determined that the driver D is in a state of low readiness for driving, and is in a state of not giving necessary attention to driving, but unlike this, in the case where step S314 is executed, it has been determined that the driver D is in a state of giving necessary attention to driving. For this reason, in step S314, the control unit 11 waits for a longer time period than in step S312. After waiting for the second time period, the control unit 11 advances the processing to step S315. Note that as long as it is longer than the first time period, the specific value of the second time period may be set as appropriate depending on the implementation.

Steps S315 to S319

Steps S315 to S319 are similar to steps S302 to S306 described above. In other words, as a result of the processing in steps S315 to S319, the control unit 11 obtains the attention state information 1251 and the readiness information 1252 from the neural network 5. Upon obtaining the attention state information 1251 and the readiness information 1252, the control unit 11 advances the processing to step S320.

Step S320

In step S320, whether or not the driver D is giving necessary attention to driving is determined based on the attention state information 1251 obtained in step S319. If the attention state information 1251 indicates that the driver D is not giving necessary attention to driving, this means that it was not possible to ensure that the driver D is giving necessary attention to driving. In this case, the control unit 11 advances the processing to step S321 in order to stop the automatic driving.

However, if the attention state information 1251 indicates that the driver D is giving necessary attention to driving, this means that it is possible to ensure that the driver D is giving necessary attention to driving. In this case, the control unit 11 returns the processing to step S302 and continues to monitor the driver D while performing the automatic driving of the vehicle.

Steps S321 to S323

In step S321, the control unit 11 defines a stopping section for safely stopping the vehicle by referencing the map information, surrounding information, and GPS information. In subsequent step S322, the control unit 11 gives an alert to inform the driver D that the vehicle is to be stopped. In subsequent step S323, the control unit 11 automatically stops the vehicle in the defined stopping section. The control unit thus ends the automatic driving processing procedure according to the present variation.

As described above, the automatic driving assist apparatus 1 maybe configured to ensure that at least the driver D is giving necessary attention to driving when controlling the automatic driving of the vehicle. In other words, when determining whether or not the driver D is in a state suited to driving the vehicle, the attention state information 1251 may be given priority over the readiness information 1252 (as a factor for determining whether or not to continue the automatic driving in the present variation). Accordingly, it is possible to estimate multiple levels of states of the driver D, and accordingly control the automatic driving. Note that the prioritized information may be the readiness information 1252 instead of the attention state information 1251.

<4.4>

In the above embodiment, the automatic driving assist apparatus 1 obtains the attention state information 1251 and the readiness information 1252 as the driving concentration information 125 in step S105. However, the driving concentration information 125 is not limited to the above example, and may be set as appropriate depending on the implementation.

For example, either the attention state information 1251 or the readiness information 1252 may be omitted. In this example, the control unit 11 may determine whether the driver D is in a state suited to driving the vehicle based on the attention state information 1251 or the readiness information 1252 in step S106 described above.

Also, the driving concentration information 125 may include information other than the attention state information 1251 and the readiness information 1252, for example. For example, the driving concentration information 125 may include information that indicates whether or not the driver D is in the driver seat, information that indicates whether or not the driver D's hands are placed on the steering wheel, information that indicates whether or not the driver D's foot is on the pedal, or the like.

Also, in the driving concentration information 125, the degree of driving concentration of the driver D itself may be expressed by a numerical value, for example. In this example, the control unit 11 may determine whether the driver D is in a state suited to driving the vehicle based on whether or not the numerical value indicated by the driving concentration information 125 is higher than a predetermined threshold in step S106 described above.

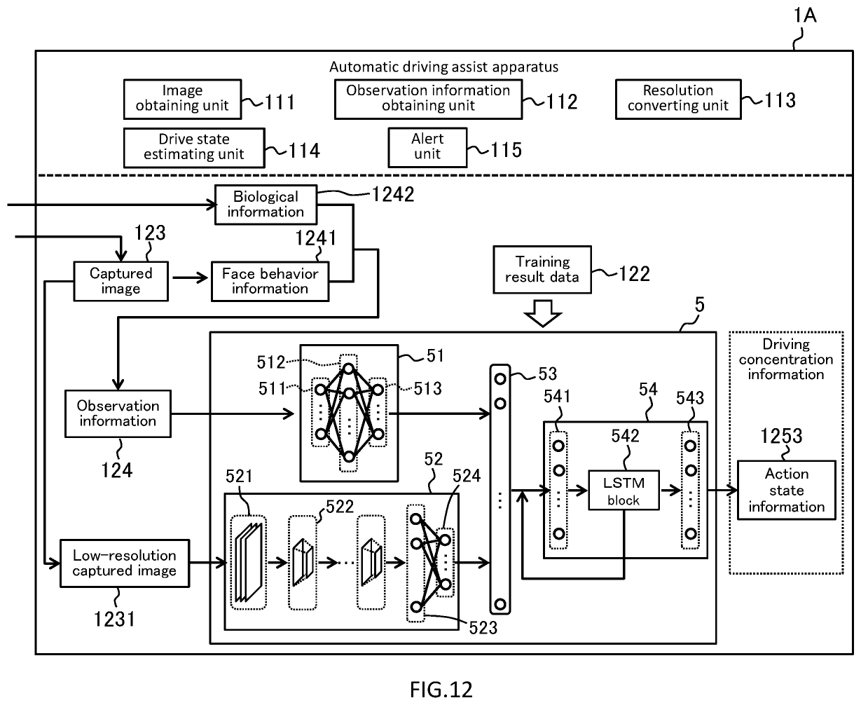

Also, as shown in FIG. 12, in step S105, the automatic driving assist apparatus 1 may obtain, as the driving concentration information 125, action state information that indicates the action state of the driver D from among a plurality of predetermined action states that have been set in correspondence with various degrees of driving concentration of the driver D.

FIG. 12 schematically illustrates an example of the function configuration of an automatic driving assist apparatus 1A according to the present variation. The automatic driving assist apparatus 1A has the same configuration as the automatic driving assist apparatus 1, with the exception that action state information 1253 is obtained as output from the neural network 5. The predetermined action states that can be estimated for the driver D may be set as appropriate depending on the implementation. For example, similarly the embodiment described above, the predetermined action states may be set as “gazing forward”, “checking meters”, “checking navigation system”, “smoking”, “eating/drinking”, “making a call”, “looking askance”, “turning around”, “drowsy”, “sleeping”, “operating mobile phone”, and “panicking”. Accordingly, through the processing of steps S101 to S105, the automatic driving assist apparatus 1A according to the present variation can estimate the action state of the driver D.

Note that in the case where the action state information 1253 is obtained as the driver concentration information, the automatic driving assist apparatus 1A may obtain the attention state information 1251 and the readiness information 1252 by specifying the attention state of the driver D and the degree of readiness for driving based on the action state information 1253. The criteria shown in FIGS. 5A and 5B or 9A and 9B can be used when specifying the attention state of the driver D and the degree of readiness for driving. In other words, in step S105, after obtaining the action state information 1253, the control unit 11 of the automatic driving assist apparatus 1A may specify the attention state of the driver D and the degree of readiness for driving in accordance with the criteria shown in FIGS. 5A and 5B or 9A and 9B. In this case, if the action state information 1253 indicates “smoking” for example, the control unit 11 can specify that the driver is giving necessary attention to driving, and is in a state of low readiness for driving.

<4.5>

In the above embodiment, the low-resolution captured image 1231 is input to the neural network 5 in step S104 described above. However, the captured image to be input to the neural network 5 is not limited to the above example. The control unit 11 may input the captured image 123 obtained in step S101 directly to the neural network 5. In this case, step S103 may be omitted from the procedure. Also, the resolution converting unit 113 may be omitted from the function configuration of the automatic driving assist apparatus 1.

Also, in the above embodiment, the control unit 11 obtains the observation information 124 in step S102, and thereafter executes processing for lowering the resolution of the captured image 123 in step S103. However, the order of processing in steps S102 and S103 is not limited to this example, and a configuration is possible in which the processing of step S103 is executed first, and then the control unit 11 executes the processing of step S102.

<4.6>

In the above embodiment, the neural network used to estimate the degree of driving concentration of the driver D includes the fully connected neural network, the convolutional neural network, the connection layer, and the LSTM network as shown in FIGS. 4 and 6. However, the architecture of the neural network used to estimate the degree of driving concentration of the driver D is not limited to the above example, and may be determined as appropriate depending on the implementation. For example, the LSTM network may be omitted.

<4.7>

In the above embodiment, a neural network is used as a learner used for estimating the degree of driving concentration of the driver D. However, as long as the learner can use the observation information 124 and the low-resolution captured image 1231 as input, the learner is not limited to being a neural network, the learner may be selected as appropriate depending on the implementation. Examples of the learner include a support vector machine, a self-organizing map, and a learner trained by reinforcement learning.

<4.8>

In the above embodiment, the control unit 11 inputs the observation information 124 and the low-resolution captured image 1231 to the neural network 5 in step S104. However, there is no limitation to this example, and information other than the observation information 124 and the low-resolution captured image 1231 may also be input to the neural network 5.

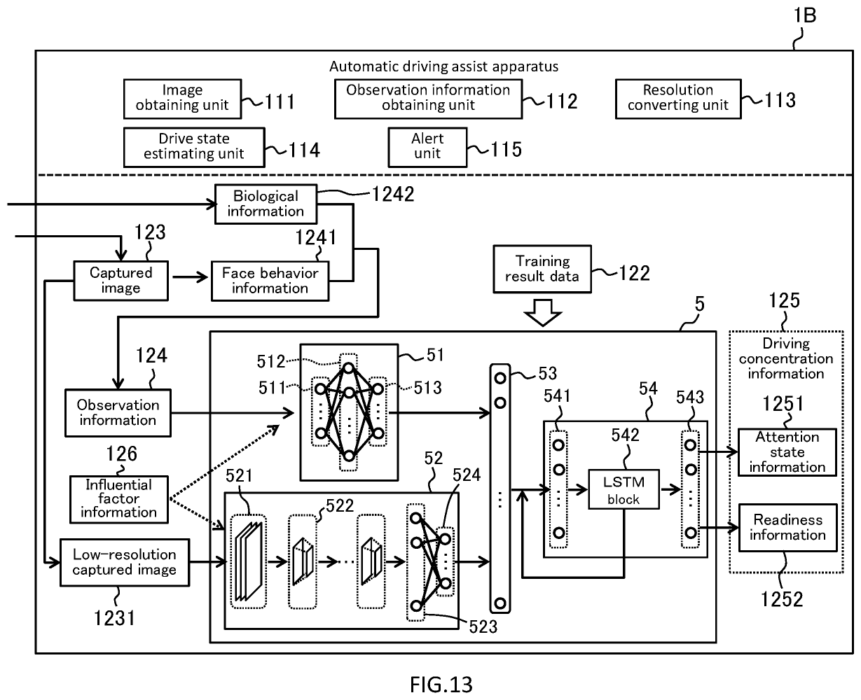

FIG. 13 schematically illustrates an example of the function configuration of an automatic driving assist apparatus 1B according to the present variation. The automatic driving assist apparatus 1B has the same configuration as the automatic driving assist apparatus 1, with the exception that influential factor information 126 regarding a factor that influences the degree of concentration of the driver D on driving is input to the neural network 5. The influential factor information 126 includes, for example, speed information indicating the traveling speed of the vehicle, surrounding environment information indicating the situation in the surrounding environment of the vehicle (measurement results from a radar device and images captured by a camera), and weather information indicating weather.

If the influential factor information 126 is indicated by numerical value data, the control unit 11 of the automatic driving assist apparatus 1B may input the influential factor information 126 to the fully connected neural network 51 of the neural network 5 in step S104. Also, if the influential factor information 126 is indicated by image data, the control unit 11 may input the influential factor information 126 to the convolutional neural network 52 of the neural network 5 in step

S104.

In this variation, the influential factor information 126 is used in addition to the observation information 124 and the low-resolution captured image 1231, thus making it possible to give consideration to a factor that influences the degree of driving concentration of the driver D when performing the estimation processing described above. The apparatus according to the present variation thus increases the accuracy of estimating the degree of driving concentration of the driver D.

Note that the control unit 11 may change the determination criterion used in step S106 based on the influential factor information 126. For example, if the attention state information 1251 and the readiness information 1252 are indicated by score values as in the variation described in 4.2, the control unit 11 may change the threshold used in the determination performed in step S106 based on the influential factor information 126. In one example, for a vehicle traveling at a higher speed as indicated by speed information, the control unit 11 may use a higher threshold value to determine that the driver D is in a state suited to driving the vehicle.

Note that the observation information 124 includes the biological information 1242 in addition to the facial behavior information 1241 in the above embodiment. However, the configuration of the observation information 124 is not limited to this example, and may be selected as appropriate depending on the embodiment. For example, the biological information 1242 may be omitted. Also, the observation information 124 may include information other than the biological information 1242, for example.

Appendix 1

A driver monitoring apparatus includes:

a hardware processor, and

a memory holding a program to be executed by the hardware processor,

the hardware processor being configured to, by executing the program, execute:

an image obtaining step of obtaining a captured image from an imaging apparatus arranged so as to capture an image of a driver seated in a driver seat of a vehicle;

an observation information obtaining step of obtaining observation information regarding the driver, the observation information including facial behavior information regarding behavior of a face of the driver; and

an estimating step of inputting the captured image and the observation information to a trained learner that has been trained to estimate a degree of concentration of the driver on driving, and obtaining, from the learner, driving concentration information regarding the degree of concentration of the driver on driving.

Appendix 2

A driver monitoring method includes:

an image obtaining step of, with use of a hardware processor, obtaining a captured image from an imaging apparatus arranged so as to capture an image of a driver seated in a driver seat of a vehicle;

an observation information obtaining step of, with use of the hardware processor, obtaining observation information regarding the driver, the observation information including facial behavior information regarding behavior of a face of the driver; and

an estimating step of, with use of the hardware processor, inputting the captured image and the observation information to a trained learner that has been trained to estimate a degree of concentration of the driver on driving, and obtaining, from the learner, driving concentration information regarding the degree of concentration of the driver on driving.

Appendix 3

A learning apparatus includes

a hardware processor, and

a memory holding a program to be executed by the hardware processor,

the hardware processor being configured to, by executing the program, execute:

a training data obtaining step of obtaining, as training data, a set of a captured image obtained from an imaging apparatus arranged so as to capture an image of a driver seated in a driver seat of a vehicle, observation information that includes facial behavior information regarding behavior of a face of the driver, and driving concentration information regarding a degree of concentration of the driver on driving; and

a learning processing step of training a learner to output an output value that corresponds to the driving concentration information when the captured image and the observation information are input.

Appendix 4

A learning method includes:

a training data obtaining step of, with use of a hardware processor, obtaining, as training data, a set of a captured image obtained from an imaging apparatus arranged so as to capture an image of a driver seated in a driver seat of a vehicle, observation information that includes facial behavior information regarding behavior of a face of the driver, and driving concentration information regarding a degree of concentration of the driver on driving; and

a learning processing step of, with use of the hardware processor, training a learner to output an output value that corresponds to the driving concentration information when the captured image and the observation information are input.

### List of Reference Numerals

1 automatic driving assist apparatus,

11 control unit, 12 storage unit, 13 external interface,

111 image obtaining unit, 112 observation information obtaining unit,

113 resolution converting unit, 114 drive state estimating unit,

115 alert unit,

121 program, 122 training result data,

123 captured image, 1231 low-resolution captured image,

124 observation information, 1241 facial behavior information, 1242 biological information,

125 driving concentration information,

1251 attention state information, 1252 readiness information,

2 learning apparatus,

21 control unit, 22 storage unit, 23 communication interface,

24 input device, 25 output device, 26 drive,

211 training data obtaining unit, 212 learning processing unit,

221 learning program, 222 training data,

223 low-resolution captured image, 224 observation information,

2251 attention state information, 2252 readiness information,

30 navigation device, 31 camera, 32 biosensor,

33 speaker,

5 neural network,

51 fully connected neural network,

511 input layer, 512 intermediate layer (hidden layer),

513 output layer,

52 convolutional neural network,

521 convolutional layer, 522 pooling layer,

523 fully connected layer, 524 output layer,

53 connection layer,

54 LSTM network (recurrent neural network),

541 input layer, 542 LSTM block, 543 output layer,

6 neural network,

61 fully connected neural network,

62 convolutional neural network, 63 connection layer,

64 LSTM network,

92 storage medium

1. A driver monitoring apparatus comprising:
an image obtaining unit configured to obtain a captured image from an imaging apparatus arranged so as to capture an image of a driver seated in a driver seat of a vehicle;
an observation information obtaining unit configured to obtain observation information regarding the driver, the observation information including facial behavior information regarding behavior of a face of the driver; and
a driver state estimating unit configured to obtain driving concentration information from a trained learner by inputting the captured image and the observation information to the trained learner and executing computational processing of the trained learner, the driving concentration information regarding a degree of concentration of the driver on driving, the captured image and the observation information obtained by the image obtaining unit and the observation information obtaining unit, wherein the trained learner has been trained by machine learning, which is for estimating the degree of concentration of the driver on driving, so as to output an output value corresponding to the driving concentration information when the captured image and the observation information are input.

2. The driver monitoring apparatus according to claim 1,
wherein the driver state estimating unit obtains, as the driving concentration information, attention state information that indicates an attention state of the driver and readiness information that indicates a degree of readiness for driving of the driver.

3. The driver monitoring apparatus according to claim 2,
wherein the attention state information indicates the attention state of the driver in a plurality of levels, and
the readiness information indicates the degree of readiness for driving of the driver in a plurality of levels.

4. The driver monitoring apparatus according to claim 3, further comprising:
an alert unit configured to alert the driver to enter a state suited to driving the vehicle in a plurality of levels in accordance with a level of the attention state of the driver indicated by the attention state information and a level of the readiness for driving of the driver indicated by the readiness information.

5. The driver monitoring apparatus according to claim 1,
wherein the driver state estimating unit obtains, as the driving concentration information, action state information that indicates an action state of the driver from among a plurality of predetermined action states that are each set in correspondence with a degree of concentration of the driver on driving.

6. The driver monitoring apparatus according to claim 1,
wherein the observation information obtaining unit obtains, as the facial behavior information, information regarding at least one of whether or not the face of the driver was detected, a face position, a face orientation, a face movement, a gaze direction, a position of a facial organ, and an eye open/closed state, by performing predetermined image analysis on the captured image that was obtained.

7. The driver monitoring apparatus according to claim 1, further comprising:
a resolution converting unit configured to lower a resolution of the obtained captured image to generate a low-resolution captured image,
wherein the driver state estimating unit inputs the low-resolution captured image to the learner.

8. The driver monitoring apparatus according to claim 1,
wherein the learner includes a fully connected neural network to which the observation information is input, a convolutional neural network to which the captured image is input, and a connection layer that connects output from the fully connected neural network and output from the convolutional neural network.

9. The driver monitoring apparatus according to claim 8,
wherein the learner further includes a recurrent neural network to which output from the connection layer is input.

10. The driver monitoring apparatus according to claim 9,
wherein the recurrent neural network includes a long short-term memory block.

11. The driver monitoring apparatus according to claim 1,
wherein the driver state estimating unit further inputs, to the learner, influential factor information regarding a factor that influences the degree of concentration of the driver on driving.

12. A driver monitoring method that causes a computer to execute:
an image obtaining step of obtaining a captured image from an imaging apparatus arranged so as to capture an image of a driver seated in a driver seat of a vehicle;
an observation information obtaining step of obtaining observation information regarding the driver, the observation information including facial behavior information regarding behavior of a face of the driver; and
an estimating step of obtaining driving concentration information from a trained learner by inputting the captured image and the observation information to the trained learner and executing computational processing of the trained learner, the driving concentration information regarding a degree of concentration of the driver on driving, the captured image and the observation information obtained by the image obtaining unit and the observation information obtaining unit, wherein the trained learner has been trained by machine learning, which is for estimating the degree of concentration of the driver on driving, so as to output an output value corresponding to the driving concentration information when the captured image and the observation information are input.

13. The driver monitoring method according to claim 12,
wherein in the estimating step, the computer obtains, as the driving concentration information, attention state information that indicates an attention state of the driver and readiness information that indicates a degree of readiness for driving of the driver.

14. The driver monitoring method according to claim 13,
wherein the attention state information indicates the attention state of the driver in a plurality of levels, and
the readiness information indicates the degree of readiness for driving of the driver in a plurality of levels.

15. The driver monitoring method according to claim 14,
wherein the computer further executes an alert step of alerting the driver to enter a state suited to driving the vehicle in a plurality of levels in accordance with a level of the attention state of the driver indicated by the attention state information and a level of the readiness for driving of the driver indicated by the readiness information.

16. The driver monitoring method according to claim 12,
wherein in the estimating step, the computer obtains, as the driving concentration information, action state information that indicates an action state of the driver from among a plurality of predetermined action states that are each set in correspondence with a degree of concentration of the driver on driving.

17. The driver monitoring method according to claim 12,
wherein in the observation information obtaining step, the computer obtains, as the facial behavior information, information regarding at least one of whether or not the face of the driver was detected, a face position, a face orientation, a face movement, a gaze direction, a position of a facial organ, and an eye open/closed state, by performing predetermined image analysis on the captured image that was obtained in the image obtaining step.

18. The driver monitoring method according to claim 12,
wherein the computer further executes a resolution converting step of lowering a resolution of the obtained captured image to generate a low-resolution captured image, and
in the estimating step, the computer inputs the low-resolution captured image to the learner.

19. The driver monitoring method according to claim 12,
wherein the learner includes a fully connected neural network to which the observation information is input, a convolutional neural network to which the captured image is input, and a connection layer that connects output from the fully connected neural network and output from the convolutional neural network.

20. The driver monitoring method according to claim 19,
wherein the learner further includes a recurrent neural network to which output from the connection layer is input.

21. The driver monitoring method according to claim 20,
wherein the recurrent neural network includes a long short-term memory block.

22. The driver monitoring method according to claim 12,
wherein in the estimating step, the computer further inputs, to the learner, influential factor information regarding a factor that influences the degree of concentration of the driver on driving.

23. A learning apparatus comprising:
a training data obtaining unit configured to obtain, as training data, a set of a captured image obtained from an imaging apparatus arranged so as to capture an image of a driver seated in a driver seat of a vehicle, observation information that includes facial behavior information regarding behavior of a face of the driver, and driving concentration information regarding a degree of concentration of the driver on driving; and
a learning processing unit configured to train a learner by machine learning to output an output value that corresponds to the driving concentration information when the captured image and the observation information are input.

24. A learning method causing a computer to execute:
a training data obtaining step of obtaining, as training data, a set of a captured image obtained from an imaging apparatus arranged so as to capture an image of a driver seated in a driver seat of a vehicle, observation information that includes facial behavior information regarding behavior of a face of the driver, and driving concentration information regarding a degree of concentration of the driver on driving; and
a learning processing step of training a learner by machine learning to output an output value that corresponds to the driving concentration information when the captured image and the observation information are input.
